(19) United States
(12) Patent Application Publication
(10) Pub. No.: US 2017/0304124 A1
(43) Pub. Date: Oct. 26, 2017

(54) Apparatuses and Methods for Folding Absorbent Articles

(71) Applicant: The Procter & Gamble Company, Cincinnati, OH (US)

(72) Inventors: Todd Douglas Lenser, Liberty Township, OH (US); Matthew Alexander Gittings, Cincinnati, OH (US)

(21) Appl. No.: 15/489,802

(22) Filed: Apr. 18, 2017

Related U.S. Application Data
(60) Provisional application No. 62/324,954, filed on Apr. 20, 2016.

(51) Int. Cl. A61F 13/15

(52) U.S. Cl. CPC A61F 13/15747; A61F 13/15764

(57) ABSTRACT

Methods and apparatuses herein relate to folding a substrate advancing in a machine direction through and assembly process, wherein the substrate includes a first outer region separated from a second outer region in a cross direction by a central region. The first outer region and the second outer region are continuous in the machine direction and the central region is discontinuous in the machine direction. During the folding process, the first outer region is conveyed in the machine direction while the central region is folded around a folding axis to place the second outer region in a facing relationship with the first outer region. The folding axis may be defined by a rail and/or a longitudinal edge of a conveyor. As such, the folding apparatus may be configured in various ways to help reduce negative effects of frictional forces acting on the central region of the substrate while folding.

### FIELD OF THE INVENTION

The present disclosure relates to methods for manufacturing absorbent articles, and more particularly, to apparatuses and methods for folding a continuous length of absorbent articles.

### BACKGROUND OF THE INVENTION

Along an assembly line, various types of articles, such as for example, diapers and other absorbent articles, may be assembled by adding components to and/or otherwise modifying an advancing, continuous web of material. For example, in some processes, advancing webs of material are combined with other advancing webs of material. In other examples, individual components created from advancing webs of material are combined with advancing webs of material, which in turn, are then combined with other advancing webs of material. In some cases, individual components created from advancing web or webs are combined with other individual components created from other advancing web or webs. Webs of material and component parts used to manufacture diapers may include: backsheets, topsheets, leg cuffs, waist bands, absorbent core components, front and/or back ears, fastening components, and various types of elastic webs and components such as leg elastics, barrier leg cuff elastics, stretch side panels, and waist elastics. Once the desired component parts are assembled, the advancing web(s) and component parts are subjected to a final knife cut to separate the web(s) into discrete diapers or other absorbent articles.

In some converting configurations, discrete absorbent chassis are arranged with a longitudinal axis parallel with a cross direction. The discrete absorbent chassis are spaced apart from each other in a machine direction and opposing waist regions of discrete absorbent chassis are then connected with continuous lengths of front and back belt webs advancing in the machine direction, forming a continuous length of absorbent articles. The continuous length of absorbent articles may then be folded in a cross direction CD. For example, Figure A shows a continuous length of diapers 10 being folded around a bar 11 while advancing in a machine direction MD. During the folding process shown in Figure A, absorbent chassis 12 may be folded around the bar 11 as a front belt web 13 and a back belt web 14 are placed into a facing relationship with the each other.

However, with continued reference to Figure A, as the folded absorbent chassis 12 advance along the bar 11 in the machine direction MD during the folding process, friction between the bar 11 and chassis 12 creates forces that act on folded portions 15 of the chassis 12 in a direction 16 that is opposite of the machine direction MD and upstream relative to the advancing front and back belt webs 13, 14. In turn, the frictional forces acting on the folded diapers 10 advancing along the bar 11 cause the shapes of the folded diapers to become askew and/or asymmetrical relative a longitudinal axis LA extending in the cross direction CD. In turn, discrete diapers that are cut from the continuous length of diapers 10 may also be misshaped. For example, Figure B shows a misshaped diaper 17 that may have been cut from the continuous length of diapers 10 in Figure A. As such, the front belt web 13, back belt web 14, and the folded portion 15 of the chassis 12 of Figure A may correspond with a front waist region 18, a back waist region 19, and a crotch region 20 of the discrete diaper 17 shown in Figure B. As shown in Figure B, the crotch region 20 of the diaper 17 is depicted as being offset, askew, or shifted laterally toward a right side of the longitudinal axis LA, defining an asymmetrical shape.

Such asymmetrically shaped diapers may be undesirable and may create various problems. For example, the asymmetrically shaped diapers may proceed to additional final forming processes during manufacture, such as folding and/or packaging operations. Figures C and D show examples of how asymmetrically shaped diapers 17 may be folded before being placed into packages, wherein the crotch region 20 of the diaper 17 continues to be offset, askew, or shifted laterally toward the right side of the longitudinal axis LA. While advancing through final forming operations, the misshaped folded portions of diapers may cause the diapers to shift, jam, and/or otherwise interfere with folding, stacking, and packaging operations. In turn, improperly packaged articles, unintended line shutdowns, and/or process equipment damage may result. In addition, stacks of asymmetrically shaped diapers may have a cumulative effect that leads to deformed and/or unstable packages. In addition, the asymmetrically shaped diapers may not present an aesthetically pleasing appearance to the consumer once removed from the package.

Consequently, it would be beneficial to provide a method and apparatus that mitigates unintended misshaping of absorbent articles during the folding process.

### SUMMARY OF THE INVENTION

The present disclosure relates to methods and apparatuses for folding a substrate advancing in a machine direction through and assembly process, wherein the substrate includes a first outer region separated from a second outer region in a cross direction by a central region. The first outer region and the second outer region are continuous in the machine direction and the central region is discontinuous in the machine direction. During the folding process, the first outer region is conveyed in the machine direction while the central region is folded around a folding axis to place the second outer region in a facing relationship with the first outer region. The folding axis may be completely or partially defined by a rail and/or a longitudinal edge of a conveyor. As such, the folding apparatus may be configured in various ways to help reduce the negative effects of frictional forces acting on the central region of the substrate during the folding process.

In one form, a method of folding absorbent articles comprises the steps of: advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction; conveying the first outer region from an upstream end region of a first conveyor to a downstream end region of the first conveyor, the first conveyor comprising a first longitudinal edge separated from a second longitudinal edge in the cross direction; partially folding the central region of the substrate along the first longitudinal edge of the first conveyor; advancing the first surface of the partially folded central region to a rail adjacent the first longitudinal edge of the first conveyor, the rail extending for a length in the machine direction; conveying the partially folded central region of the substrate with a second conveyor, wherein the second conveyor comprises a carrier surface comprising an upstream end region angularly offset from a downstream end region to define a generally helical-shaped conveyance path along the machine direction, the carrier surface contacting the second surface of the central region while advancing the first surface of the central region along the length of the rail; and folding the central region around the rail to position the second outer region into a facing relationship with the first outer region.

In another form, a method of folding absorbent articles comprises the steps of: advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction; conveying the first outer region from an upstream end region of a first conveyor to a downstream end region of the first conveyor, wherein the first conveyor comprises a first carrier surface, the first carrier surface contacting the first surface of the first outer region of the substrate; wrapping a portion of the first surface of the central region of the substrate around a rail, the rail extending for a length in the machine direction; conveying the central region of the substrate with a second conveyor, wherein the second conveyor comprises a second carrier surface comprising an upstream end region angularly offset from a downstream end region to define a generally helical-shaped conveyance path along the machine direction, the second carrier surface contacting the second surface of the central region while advancing the wrapped portion of the substrate along the length of the rail; conveying the second outer region of the substrate with a third conveyor, wherein the third conveyor comprises a carrier surface comprising an upstream end region angularly offset from a downstream end region to define a generally helical-shaped conveyance path along the machine direction, the carrier surface contacting the second surface of the second outer region while advancing the conveying the central region of the substrate with the second conveyor; and folding the substrate around the rail to position the second outer region into a facing relationship with the first outer region.

In yet another form, a method of folding absorbent articles comprises the steps of: advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction; conveying the first outer region from an upstream end region of a first conveyor to a downstream end region of the first conveyor, wherein the first conveyor comprises a first carrier surface, the first carrier surface contacting the first surface of the first outer region of the substrate; applying a vacuum force to the central region of the substrate with a second conveyor, wherein the second conveyor comprises a second carrier surface comprising an upstream end region angularly offset from a downstream end region to define a generally helical-shaped conveyance path along the machine direction; applying a vacuum force to the second outer region of the substrate with a third conveyor, wherein the third conveyor comprises a third carrier surface comprising an upstream end region angularly offset from a downstream end region to define a generally helical-shaped conveyance path along the machine direction; folding the substrate to position the second outer region into a facing relationship with the first outer region by advancing the central region and second outer region of the substrate with the second and third conveyors in the machine direction.

### BRIEF DESCRIPTION OF THE DRAWINGS

Figure A is a view of a prior art process showing a continuous length of diapers advancing in a machine direction while being folded around a bar.

Figure B is a front plan view of an asymmetrically shaped discrete diaper from Figure A.

Figure C is a front plan view of the diaper in Figure B with additional folds.

Figure D is a front plan view of the diaper in Figure C with an additional fold.

FIG. 1A is a front perspective view of a diaper pant.

FIG. 1B is a rear perspective view of a diaper pant.

FIG. 2 is a partially cut away plan view of the diaper pant shown in FIGS. 1A and 1B in a flat, uncontracted state.

FIG. 3A is a cross-sectional view of the diaper pant of FIG. 2 taken along line 3A-3A.

FIG. 3B is a cross-sectional view of the diaper pant of FIG. 2 taken along line 3B-3B.

FIG. 4 is a schematic side view of a converting apparatus adapted to manufacture pre-fastened, pant diapers.

FIG. 5A1 is a view of a continuous length of an advancing first substrate from FIG. 4 taken along line A1-A1.

FIG. 5A2 is a view of a continuous length of an advancing elastic laminate from FIG. 4 taken along line A2-A2.

FIG. 5B is a view of continuous lengths of advancing first and second elastic belt laminates from FIG. 4 taken along line B-B.

FIG. 5C is a view of a continuous length of chassis assemblies from FIGS. 4 and 8 taken along line C-C.

FIG. 5D1 is a view of a discrete chassis from FIGS. 4 and 8 taken along line D1-D1.

FIG. 5D2 is a view of a discrete chassis from FIGS. 4 and 8 taken along line D2-D2.

FIG. 5E1 is a view of multiple discrete chassis spaced from each other along the machine direction MD and connected with each other by the first and second elastic belt laminates from FIG. 4 taken along line E1-E1.

FIG. 5E2 is a view of multiple discrete chassis spaced from each other along the machine direction MD and connected with each other by the first and second elastic belt laminates from FIG. 4 taken along line E2-E2.

FIG. 5F is a view of folded multiple discrete chassis with the first and second elastic belt laminates in a facing relationship from FIG. 4 taken along line F-F.

FIG. 5G is a view of two discrete absorbent articles advancing the machine direction MD from FIG. 4 taken along line G-G.

FIG. 6A is a front perspective view of a diaper pant constructed with a contiguous outer cover.

FIG. 6B is a front plan view of the diaper pant of FIG. 6A.

FIG. 6C is a rear plan view of the diaper pant of FIG. 6A.

FIG. 7 is a partially cut away plan view of the diaper pant shown in FIGS. 6A-6C in a flat, uncontracted state.

FIG. 8 is a schematic side view of a converting apparatus adapted to manufacture pre-fastened, pant diapers.

FIG. 9A1 is a view of a continuous length of an advancing substrate from FIG. 8 taken along line A1-A1.

FIG. 9A2 is a view of a continuous length of an advancing elastic laminate from FIG. 8 taken along line A2-A2.

FIG. 9A3 is a view of a continuous length of an advancing elastic laminate from FIG. 8 taken along line A3-A3.

FIG. 9B is a view of continuous lengths of advancing first and second elastic belt laminates from FIG. 8 taken along line B-B.

FIG. 9E1 is a view of multiple discrete chassis spaced from each other along the machine direction MD and connected with each other by an outer cover and the first and second elastic belt laminates from FIG. 8 taken along line E1-E1.

FIG. 9E2 is a view of multiple discrete chassis spaced from each other along the machine direction MD and connected with each other by an outer cover and the first and second elastic belt laminates from FIG. 8 taken along line E2-E2.

FIG. 9F is a view of folded multiple discrete chassis with the first and second elastic belt laminates in a facing relationship from FIG. 8 taken along line F-F.

FIG. 9G is a view of two discrete absorbent articles advancing the machine direction MD from FIG. 8 taken along line G-G.

FIG. 10A is an isometric side view of a substrate in the form of a continuous length of absorbent articles advancing in a machine direction MD along an embodiment of a folding apparatus.

FIG. 10B is a side view of the folding apparatus and substrate from FIG. 10A taken along line 10B-10B.

FIG. 10C is a detailed side view of the folding apparatus and substrate from FIG. 10B taken along line 10C-10C.

FIG. 10D is an isometric side view of the substrate of FIG. 10A.

FIG. 10E is an isometric side view of the folding apparatus of FIG. 10A.

FIG. 10F is a side view of the folding apparatus of FIG. 10A.

FIG. 11 is an isometric side view of a substrate in the form of a continuous length of absorbent articles advancing in a machine direction MD along a second embodiment of a folding apparatus wherein a folding axis is defined by an edge of a conveyor and a rail.

FIG. 12 is an isometric side view of a substrate in the form of a continuous length of absorbent articles advancing in a machine direction MD along a third embodiment of a folding apparatus wherein a folding axis is defined by an edge of a conveyor.

### DETAILED DESCRIPTION OF THE INVENTION

The following term explanations may be useful in understanding the present disclosure: “Absorbent article” is used herein to refer to consumer products whose primary function is to absorb and retain soils and wastes. “Diaper” is used herein to refer to an absorbent article generally worn by infants and incontinent persons about the lower torso. The term “disposable” is used herein to describe absorbent articles which generally are not intended to be laundered or otherwise restored or reused as an absorbent article (e.g., they are intended to be discarded after a single use and may also be configured to be recycled, composted or otherwise disposed of in an environmentally compatible manner).

An “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

“Longitudinal” means a direction running substantially perpendicular from a waist edge to a longitudinally opposing waist edge of an absorbent article when the article is in a flat out, uncontracted state, or from a waist edge to the bottom of the crotch, i.e. the fold line, in a bi-folded article. Directions within 45 degrees of the longitudinal direction are considered to be “longitudinal.” “Lateral” refers to a direction running from a longitudinally extending side edge to a laterally opposing longitudinally extending side edge of an article and generally at a right angle to the longitudinal direction. Directions within 45 degrees of the lateral direction are considered to be “lateral.”

The term “substrate” is used herein to describe a material which is primarily two-dimensional (i.e. in an XY plane) and whose thickness (in a Z direction) is relatively small (i.e. 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, rear waist fastened or seamed).

The present disclosure relates to methods and apparatuses for folding substrates advancing in a machine direction. Such substrates are generally described herein as continuous lengths of absorbent articles advancing in a machine direction through a folding process. As such, the substrates include a first surface and an opposing second surface, and further include a first outer region separated from a second outer region in a cross direction by a central region. The first outer region and the second outer region are continuous in the machine direction and the central region is discontinuous in the machine direction. During the folding process, the first outer region is conveyed from an upstream end region of a first conveyor to a downstream end region of the first conveyor, while the central region is folded around a folding axis to place the second outer region in a facing relationship with the first outer region. The folding axis may be completely or partially defined by a rail and/or a longitudinal edge of the first conveyor. As discussed in more detail below, the folding apparatus may be configured in various ways to help reduce the negative effects of frictional forces acting on the central region of the substrate resulting from the substrate advancing along the rail during the folding process. For example, the folding apparatus herein may include second and/or third helical or twisted conveyors that may apply vacuum pressure to the central region and/or second outer region during the folding process. As such, the vacuum pressure helps to hold the substrate against the carrier surfaces of the conveyors, which in turn, enables to the conveyors to pull the substrate in the machine direction in opposition to the friction forces acting on the substrate during the folding process. In some configurations, frictional forces acting on the substrate may also be reduced by reducing the length of the rail. As such, the folding axis may be defined in part by both a longitudinal edge of the first conveyor and the rail. And in some configurations, the folding apparatus may not include a rail, and as such, the folding axis may be completely defined by the first longitudinal edge of the first conveyor.

As previously mentioned, the processes and apparatuses discussed herein may be used to fold various types of substrate configurations, some of which may be used in the manufacture of different types of absorbent articles. To help provide additional context to the subsequent discussion of the process embodiments, the following provides a general description of absorbent articles in the form of diapers that include components that may be folded in accordance with the methods and apparatuses disclosed herein.

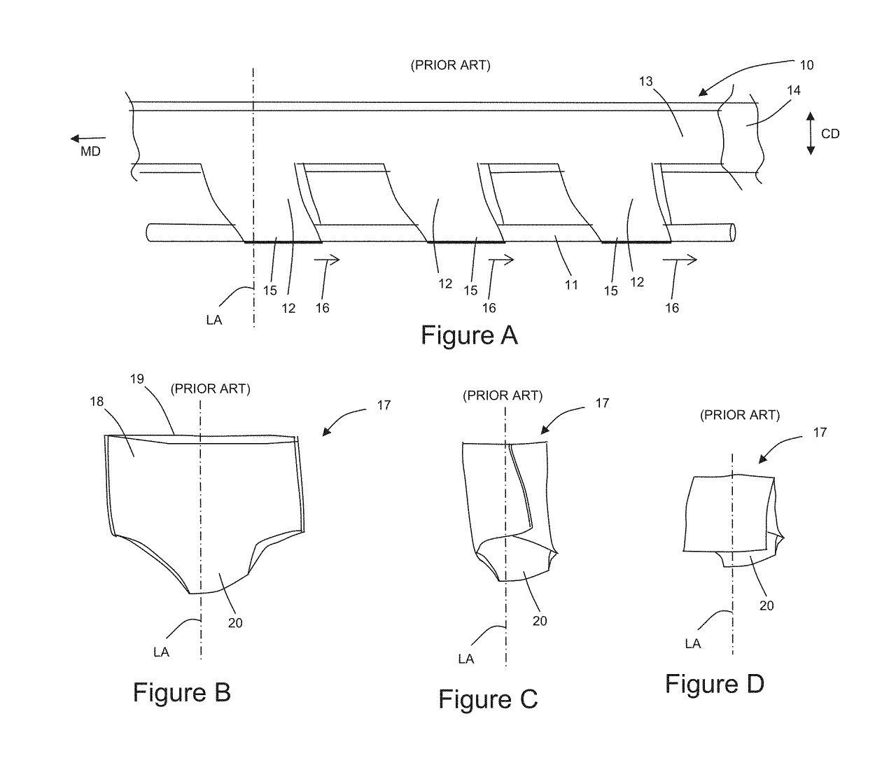

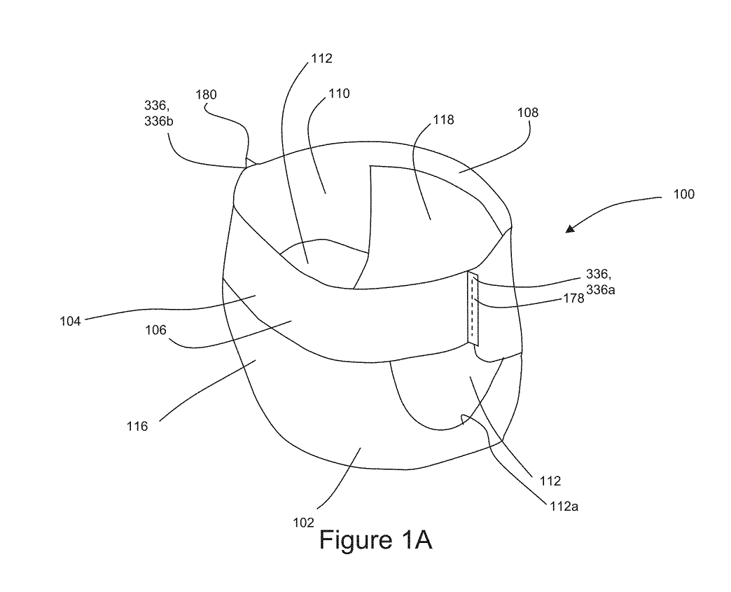

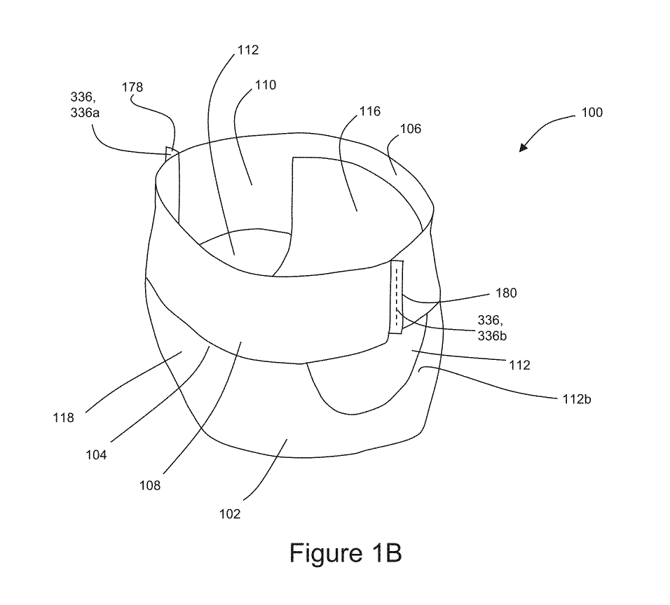

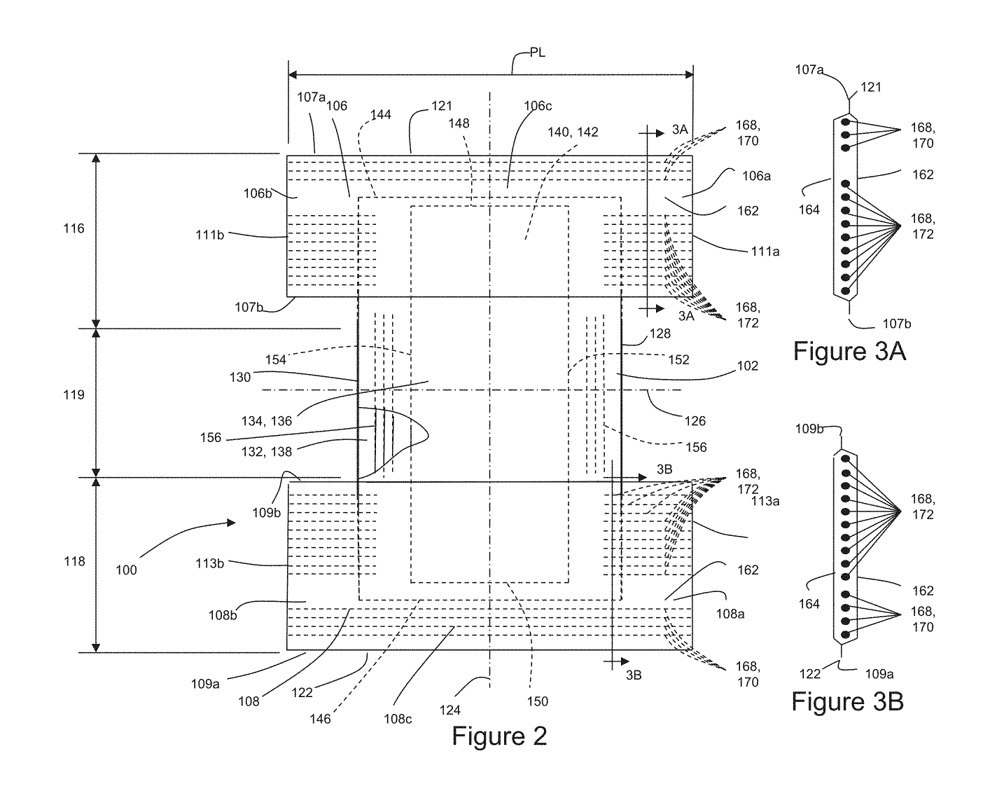

FIGS. 1A, 1B, and 2 show an example of a diaper pant 100 that may be assembled and folded in accordance with the apparatuses and methods disclosed herein. In particular, FIGS. 1A and 1B show perspective views of a diaper pant 100 in a pre-fastened configuration, and FIG. 2 show a plan view of the diaper pant 100 with the portion of the diaper that faces away from a wearer oriented toward the viewer. The diaper pant 100 includes a chassis 102 and a ring-like elastic belt 104. As discussed below in more detail, a first elastic belt 106 and a second elastic belt 108 are bonded together to form the ring-like elastic belt 104.

With continued reference to FIG. 2, the diaper pant 100 and the chassis 102 each include a first waist region 116, a second waist region 118, and a crotch region 119 disposed intermediate the first and second waist regions. The first waist region 116 may be configured as a front waist region, and the second waist region 118 may be configured as back waist region. In some embodiments, the length of each of the front waist region, back waist region, and crotch region may be ⅓ of the length of the absorbent article 100. The diaper 100 may also include a laterally extending front waist edge 121 in the front waist region 116 and a longitudinally opposing and laterally extending back waist edge 122 in the back waist region 118. To provide a frame of reference for the present discussion, the diaper 100 and chassis 102 of FIG. 2 are shown with a longitudinal axis 124 and a lateral axis 126. In some embodiments, the longitudinal axis 124 may extend through the front waist edge 121 and through the back waist edge 122. And the lateral axis 126 may extend through a first longitudinal or right side edge 128 and through a midpoint of a second longitudinal or left side edge 130 of the chassis 102.

As shown in FIGS. 1A, 1B, and 2, the diaper pant 100 may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100 may also include other features, such as leg elastics and/or leg cuffs to enhance the fit around the legs of the wearer.

As shown in FIG. 2, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 2, the laterally extending end edges 144 and 146 are located longitudinally inward from the laterally extending front waist edge 121 in the front waist region 116 and the laterally extending back waist edge 122 in the back waist region 118. When the diaper pant 100 is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the front waist region 116 through the crotch region 119 to the back waist region 118.

It is to also be appreciated that a portion or the whole of the diaper 100 may also be made laterally extensible. The additional extensibility may help allow the diaper 100 to conform to the body of a wearer during movement by the wearer. The additional extensibility may also help, for example, the user of the diaper 100, including a chassis 102 having a particular size before extension, to extend the front waist region 116, the back waist region 118, or both waist regions of the diaper 100 and/or chassis 102 to provide additional body coverage for wearers of differing size, i.e., to tailor the diaper to an individual wearer. Such extension of the waist region or regions may give the absorbent article a generally hourglass shape, so long as the crotch region is extended to a relatively lesser degree than the waist region or regions, and may impart a tailored appearance to the article when it is worn.

As previously mentioned, the diaper pant 100 may include a backsheet 136. The backsheet 136 may also define the outer surface 134 of the chassis 102. The backsheet 136 may be impervious to fluids (e.g., menses, urine, and/or runny feces) and may be manufactured in part from a thin plastic film, although other flexible liquid impervious materials may also be used. The backsheet 136 may prevent the exudates absorbed and contained in the absorbent core from wetting articles which contact the diaper 100, such as bedsheets, pajamas and undergarments. The backsheet 136 may also comprise a woven or nonwoven material, polymeric films such as thermoplastic films of polyethylene or polypropylene, and/or a multi-layer or composite materials comprising a film and a nonwoven material (e.g., having an inner film layer and an outer nonwoven layer). The backsheet may also comprise an elastomeric film. An example backsheet 136 may be a polyethylene film having a thickness of from about 0.012 mm (0.5 mils) to about 0.051 mm (2.0 mils). Exemplary polyethylene films are manufactured by Clopay Corporation of Cincinnati, Ohio, under the designation BR-120 and BR-121 and by Tredegar Film Products of Terre Haute, Ind., under the designation XP-39385. The backsheet 136 may also be embossed and/or matte-finished to provide a more clothlike appearance. Further, the backsheet 136 may permit vapors to escape from the absorbent core (i.e., the backsheet is breathable) while still preventing exudates from passing through the backsheet 136. The size of the backsheet 136 may be dictated by the size of the absorbent core 142 and/or particular configuration or size of the diaper 100.

Also described above, the diaper pant 100 may include a topsheet 138. The topsheet 138 may also define all or part of the inner surface 132 of the chassis 102. The topsheet 138 may be compliant, soft feeling, and non-irritating to the wearer's skin. It may be elastically stretchable in one or two directions. Further, the topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art.

Topsheets 138 may be selected from high loft nonwoven topsheets, apertured film topsheets and apertured nonwoven topsheets. Apertured film topsheets may be pervious to bodily exudates, yet substantially non-absorbent, and have a reduced tendency to allow fluids to pass back through and rewet the wearer's skin. Exemplary apertured films may include those described in U.S. Pat. Nos. 5,628,097; 5,916,661; 6,545,197; and 6,107,539.

As mentioned above, the diaper pant 100 may also include an absorbent assembly 140 that is joined to the chassis 102. As shown in FIG. 2, the absorbent assembly 140 may have a laterally extending front edge 148 in the front waist region 116 and may have a longitudinally opposing and laterally extending back edge 150 in the back waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprises primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 and 2004/0097895.

As previously mentioned, the diaper 100 may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1.

As mentioned above, diaper pants may be manufactured with a ring-like elastic belt 104 and provided to consumers in a configuration wherein the front waist region 116 and the back waist region 118 are connected to each other as packaged, prior to being applied to the wearer. As such, diaper pants may have a continuous perimeter waist opening 110 and continuous perimeter leg openings 112 such as shown in FIGS. 1A and 1B. The ring-like elastic belt may be formed by joining a first elastic belt to a second elastic belt with a permanent side seam or with an openable and reclosable fastening system disposed at or adjacent the laterally opposing sides of the belts.

As previously mentioned, the ring-like elastic belt 104 may be defined by a first elastic belt 106 connected with a second elastic belt 108. As shown in FIG. 2, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. The distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108. The central region 106c of the first elastic belt is connected with the first waist region 116 of the chassis 102, and the central region 108c of the second elastic belt 108 is connected with the second waist region 116 of the chassis 102. As shown in FIGS. 1A and 1B, the first end region 106a of the first elastic belt 106 is connected with the first end region 108a of the second elastic belt 108 at first side seam 178, and the second end region 106b of the first elastic belt 106 is connected with the second end region 108b of the second elastic belt 108 at second side seam 180 to define the ring-like elastic belt 104 as well as the waist opening 110 and leg openings 112.

It is to be appreciated that the first and second elastic belts may define various pitch lengths PL. For example, in some embodiments, the pitch lengths PL of the first and/or second elastic belts may be about 300 mm to about 1100 mm.

As shown in FIGS. 2, 3A, and 3B, the first elastic belt 106 also defines an outer laterally extending edge 107a and an inner laterally extending edge 107b, and the second elastic belt 108 defines an outer laterally extending edge 109a and an inner laterally extending edge 109b. As such, a perimeter edge 112a of one leg opening may be defined by portions of the inner laterally extending edge 107b of the first elastic belt 106, the inner laterally extending edge 109b of the second elastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100. The first elastic belt and the second elastic belt may also each include an outer, garment facing layer 162 and an inner, wearer facing layer 164. It is to be appreciated that the first elastic belt 106 and the second elastic belt 108 may comprise the same materials and/or may have the same structure. In some embodiments, the first elastic belt 106 and the second elastic belt may comprise different materials and/or may have different structures. It should also be appreciated that the first elastic belt 106 and the second elastic belt 108 may be constructed from various materials. For example, the first and second belts may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some embodiments, the first and second elastic belts include a nonwoven web of synthetic fibers, and may include a stretchable nonwoven. In other embodiments, the first and second elastic belts include an inner hydrophobic, non-stretchable nonwoven material and an outer hydrophobic, non-stretchable nonwoven material.

The first and second elastic belts 106, 108 may also each include belt elastic material interposed between the outer substrate layer 162 and the inner substrate layer 164. The belt elastic material may include one or more elastic elements such as strands, ribbons, films, or panels extending along the lengths of the elastic belts. As shown in FIGS. 2, 3A, and 3B, the belt elastic material may include a plurality of elastic strands 168 which may be referred to herein as outer, waist elastics 170 and inner, waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. In some embodiments, some elastic strands 168, such as the inner waist elastics 172, may be configured with discontinuities in areas, such as for example, where the first and second elastic belts 106, 108 overlap the absorbent assembly 140. In some embodiments, the elastic strands 168 may be disposed at a constant interval in the longitudinal direction. In other embodiments, the elastic strands 168 may be disposed at different intervals in the longitudinal direction. The belt elastic material in a stretched condition may be interposed and joined between the uncontracted outer layer and the uncontracted inner layer. When the belt elastic material is relaxed, the belt elastic material returns to an unstretched condition and contracts the outer layer and the inner layer. The belt elastic material may provide a desired variation of contraction force in the area of the ring-like elastic belt. It is to be appreciated that the chassis 102 and elastic belts 106, 108 may be configured in different ways other than as depicted in FIG. 2. The belt elastic material may be joined to the outer and/or inner layers continuously or intermittently along the interface between the belt elastic material and the inner and/or outer belt layers.

In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168, 172 that extend along non-linear or curved paths that may correspond with the curved contours of the inner lateral edges 107b, 109b.

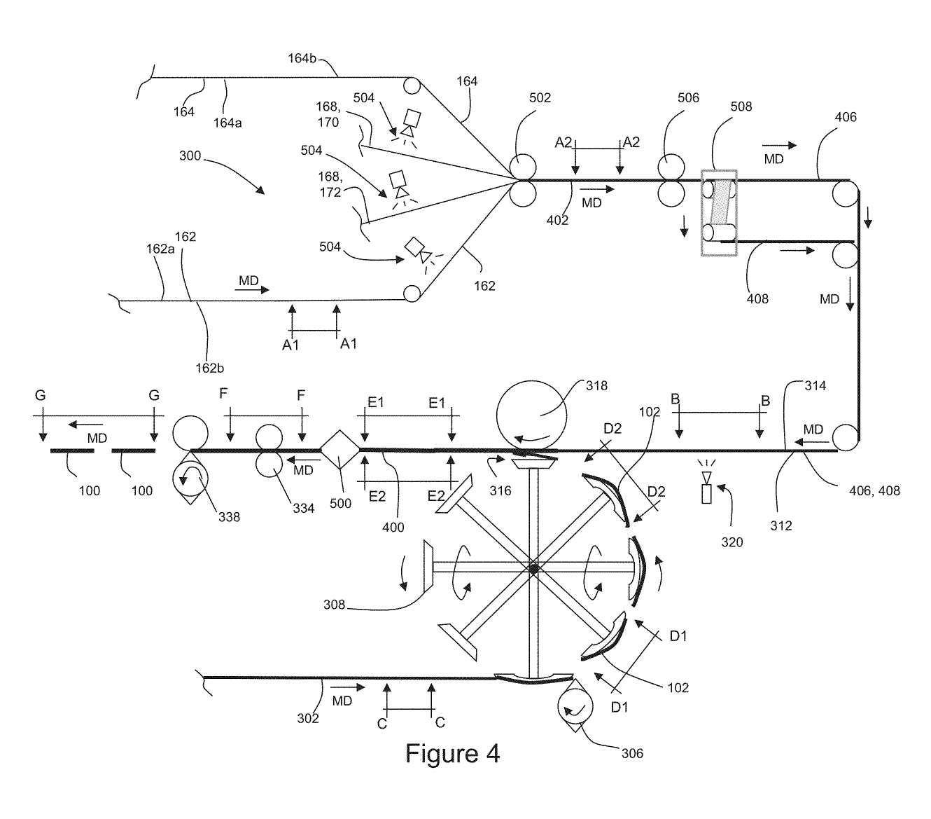

As previously mentioned, the apparatuses and methods according to the present disclosure may be utilized to assemble various components of pre-fastened, refastenable pant diapers 100. For example, FIG. 4 shows a schematic view of a converting apparatus 300 adapted to manufacture pant diapers 100. The method of operation of the converting apparatus 300 may described with reference to the various components of pant diapers 100 described above and shown in FIGS. 1A, 1B, and 2. Although the following methods are provided in the context of the diaper 100 shown in FIGS. 1A, 1B, and 2, it is to be appreciated that various embodiments of diaper pants can be manufactured according the methods disclosed herein, such as for example, the absorbent articles disclosed in U.S. Pat. No. 7,569,039; U.S. Patent Publication Nos. 2005/0107764 A1, 2012/0061016 A1, and 2012/0061015 A1, which are all hereby incorporated by reference herein.

As described in more detail below, the converting apparatus 300 shown in FIG. 4 operates to advance first and second elastic belt laminates 406, 408 along a machine direction MD. In addition, a continuous length of chassis assemblies 302 are advanced in a machine direction MD and cut into discrete chassis 102 such that the longitudinal axis 124 of each chassis 102 is parallel with the machine direction MD. The discrete chassis 102 are then turned to advance the discrete chassis 102 along the machine direction MD such that the lateral axis 126 of each chassis 102 is parallel with the machine direction MD. The discrete chassis 102 are also spaced apart from each other along the machine direction MD. Opposing waist regions 116, 118 of the spaced apart chassis 102 are then connected with continuous lengths of advancing first and second elastic belt laminates 406, 408. The chassis 102 may then be folded along the lateral axis, or parallel to the lateral axis, to bring the first and second elastic belt laminates 406, 408 into a facing relationship, and the first and second elastic belt laminates are bonded together with laterally opposing bonds 336. As discussed in more detail below, the first and second elastic belt laminates may be bonded together with adjacent bonds 336a, 336b intermittently spaced along the machine direction MD. It is to be appreciated that the bonds 336a, 336b may be configured as permanent and/or refastenable bonds. And each bond 336a, 336b may be a discrete bond site extending contiguously in a cross direction CD across a width of the first and second elastic belt laminates and/or may include a plurality of relatively small, discrete bond sites arranged in the cross direction. The first and second continuous elastic laminates 406, 408 are then cut in the cross direction CD between adjacent bonds 336a, 336b to create discrete pant diapers 100, such as shown in FIGS. 1A and 1B.

As shown in FIG. 4, a first continuous substrate layer in the form of a continuous length of outer layer belt substrate 162; a second continuous substrate layer in the form of a continuous length of inner layer belt substrate 164; and elastics 168 are combined to form a continuous elastic laminate 402 in the form of a belt material. More particularly, continuous lengths of outer layer belt substrate 162, inner layer belt substrate 164, outer elastic strands 170 and inner elastic strands 172 are advanced in a machine direction MD and combined at nip rolls 502 to form the continuous elastic laminate 402.

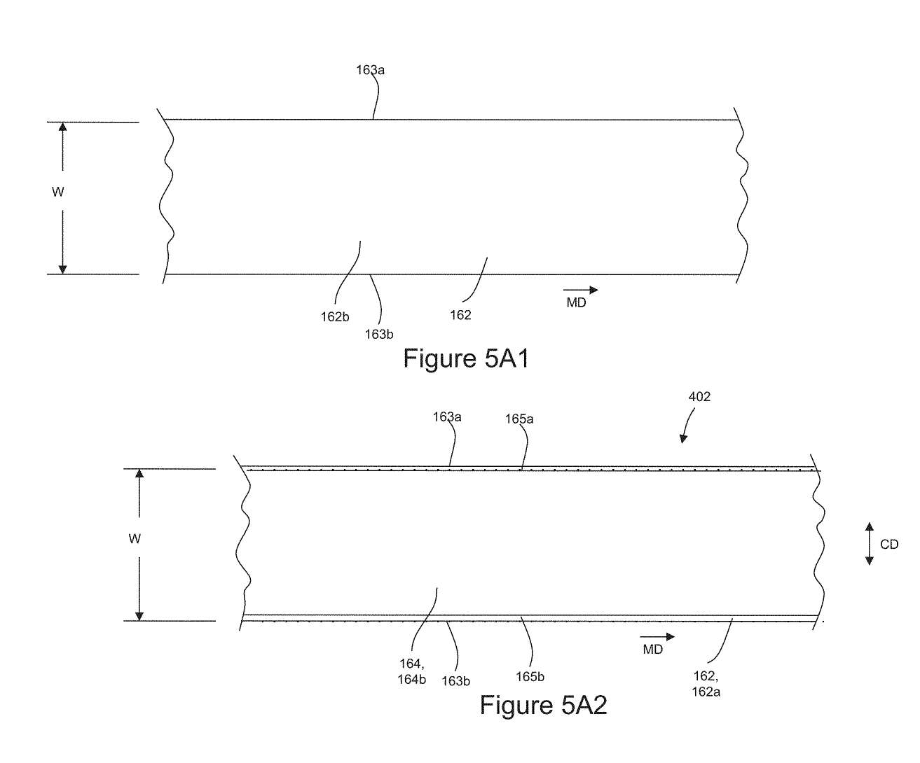

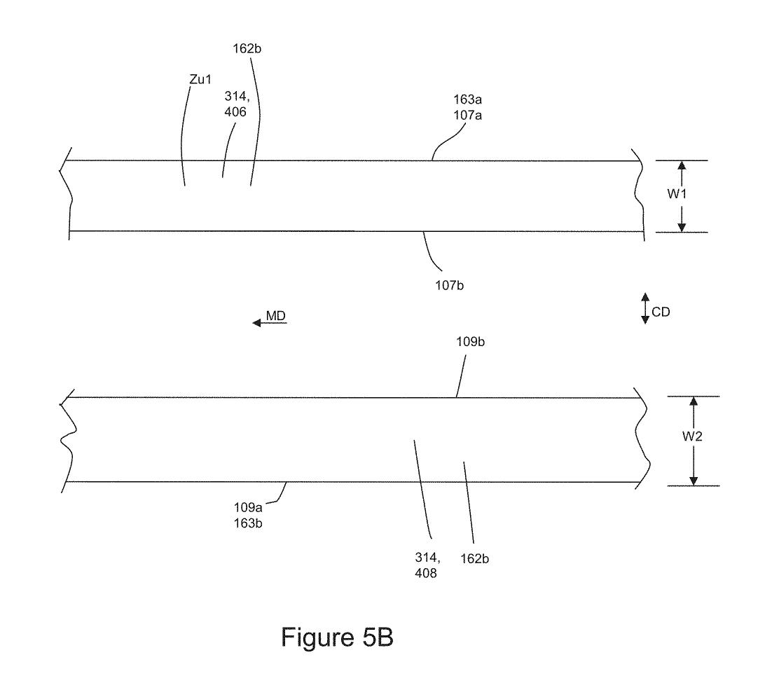

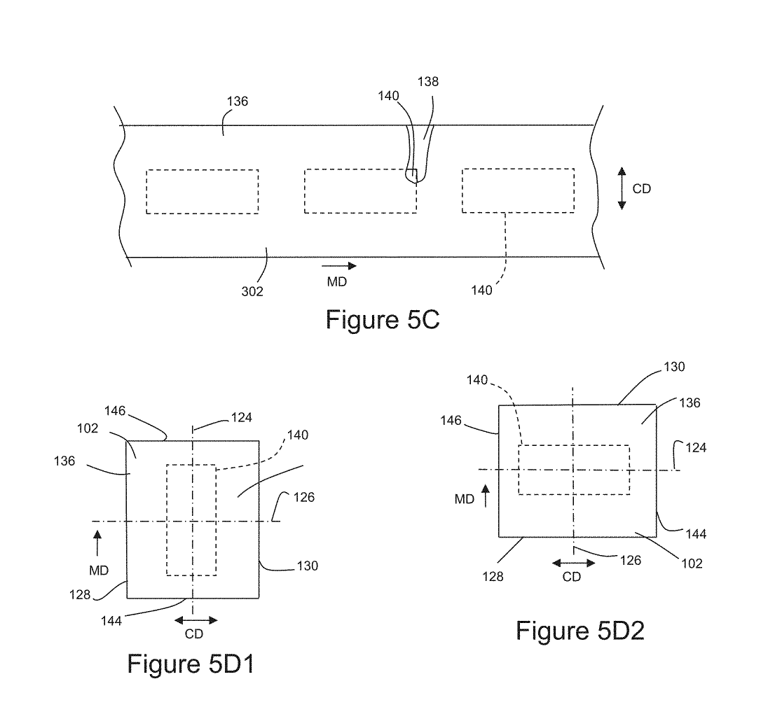

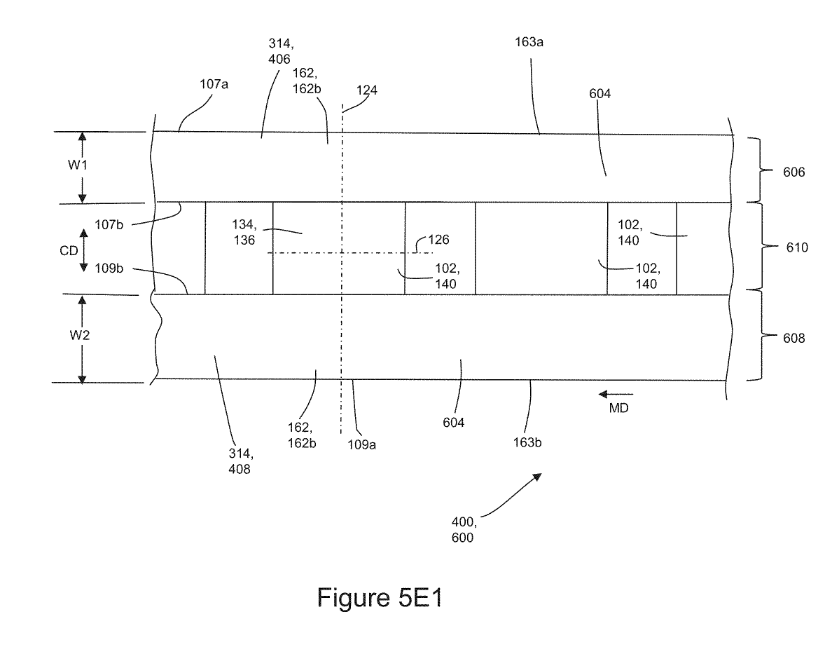

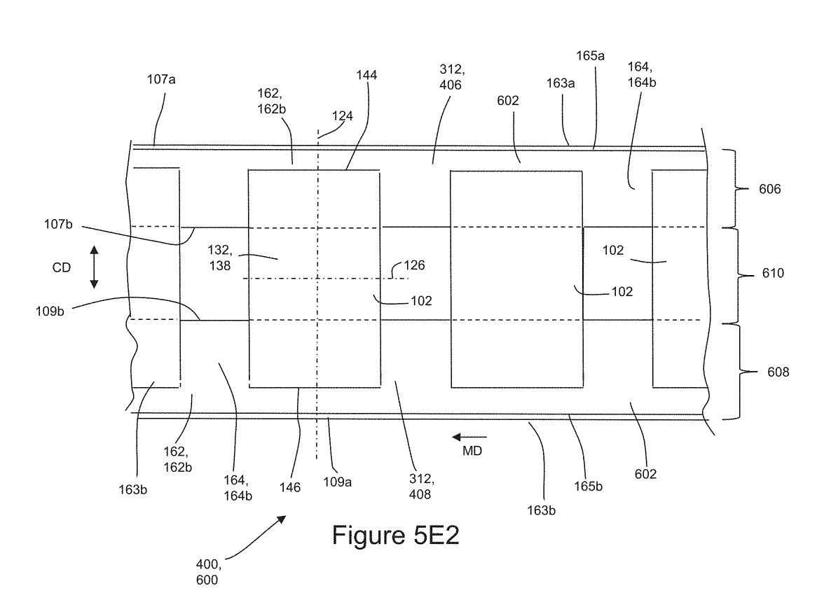

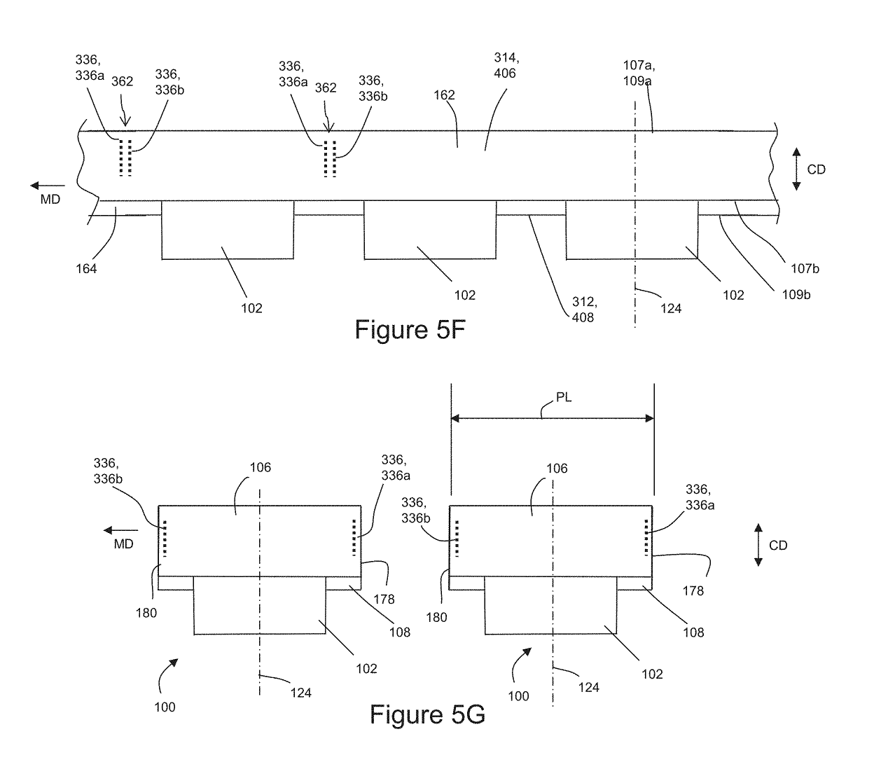

As shown in FIGS. 4, 5A1, and 5A2, the outer belt substrate 162 includes first surface 162a and an opposing second surface 162b, and defines a width W in the cross direction CD between opposing first and second longitudinal edges 163a, 163b. And the inner belt substrate 164 includes first surface 164a and an opposing second surface 164b, and defines a width in the cross direction CD between opposing first and second longitudinal edges 165a, 165b. As shown in FIG. 5A2, the width W of the outer belt substrate 162 may be greater than the width of the inner belt substrate 164. And the width W of the outer belt substrate 162 may also define the width W of the elastic laminate 402. It is to be appreciated that in some embodiments, the width of the inner belt substrate 164 may be the same as or greater than the width of the outer belt substrate 162.

With continued reference to FIG. 4, before entering the nip rolls 502, the outer elastic strands 170 and inner elastic strands 172 are stretched in the machine direction MD. In addition, adhesive 504 may be applied to the elastic strands 170, 172 as well as either or both of the continuous lengths of outer layer belt substrate 162 and inner layer belt substrate 164 before entering nip rolls 502. As such, the elastic strands 168 are bonded between the first surface 162a of the outer layer belt substrate 162 and the first surface 164a of inner layer belt substrate 164 at the nip rolls 502. Further, adhesive 504 may be applied intermittently along the lengths of the inner elastic strands 172 and/or intermittently along the length of either or both of the continuous lengths of outer layer belt substrate 162 and inner layer belt substrate 164 before entering nip rolls 502. As such, the inner elastic strands 172 may be intermittently bonded to either or both of the continuous lengths of outer layer belt substrate 162 and inner layer belt substrate 164 along the machine direction MD. Thus, the continuous elastic laminate 402 may include non-bonded regions intermittently spaced between bonded regions along the machine direction MD, wherein the inner elastic strands 172 are not bonded to either the outer layer belt substrate 162 or inner layer belt substrate 164 in the non-bonded regions. And the inner elastic strands 172 are bonded to the outer layer belt substrate 162 and/or inner layer belt substrate 164 in the bonded regions. As such, the elastic strands 172 may be severed in the non-bonded regions in a subsequent process step. Although FIG. 4 shows an embodiment wherein the continuous elastic laminate 402 is formed by combining continuous lengths of outer layer belt substrate 162 and inner layer belt substrate 164 with elastic strands 168, it is to be appreciated the continuous elastic laminate 402 can be formed in various ways, such as disclosed in U.S. Pat. No. 8,440,043 and U.S. Patent Publication Nos. 2013/0255861 A1; 2013/0255862 A1; 2013/0255863 A1; 2013/0255864 A1; and 2013/0255865 A1.

With continued reference to FIGS. 4, 5A2, and 5B, from the nip rolls 502 the continuous elastic laminate 402 advances in the machine direction MD to a cutter 506 that cuts the continuous elastic laminate 402 into two continuous elastic belt laminates, referred to as a first elastic belt laminate 406 and a second elastic belt laminate 408. In particular, the cutter 506 operates to cut the elastic laminate 402 along the machine direction to form the first continuous elastic laminate 406 and the second continuous elastic laminate 408. In some embodiments, the cutter 506 operates to cut the elastic laminate 402 along the machine direction to form the first continuous elastic laminate 406 and the second continuous elastic laminate 408. As shown in FIG. 5B, the first elastic laminate 406 includes an inner longitudinal edge 107b and an outer longitudinal edge 107a, and the second elastic laminate 406 includes an inner longitudinal edge 109b and an outer longitudinal edge 109a.

As shown in FIG. 5B, the first belt laminate 406 extends between the outer longitudinal edge 107a and the inner longitudinal edge 107b to define a width W1 in the cross direction CD. And the second belt laminate 408 extends between the outer longitudinal edge 109a and the inner longitudinal edge 109b to define a width W2 in the cross direction CD. It is to be appreciated that W2 may be greater than W1. It is also to be appreciated that in some configurations, W1 may be equal to or greater than W2. In some embodiments, the widths W1 and/or W2 may be from about 120 mm to about 300 mm.

It is also to be appreciated that the cutter 506 may be configured in various ways. For example, in some embodiments the cutter 506 may be a slitter or a die cutter that separates the belt material into two continuous belt substrates with either a straight line cut and/or a curved line cut. The cutter 506 may also be configured as a perforator that perforates the belt material with a line of weakness and wherein the belt material is separated along the line of weakness in a later step. From the cutter 506, the first and second belt laminates 406, 408 advance through a diverter 508 that separates the first and second belt substrates from each other in the cross direction CD, such as shown in FIG. 5B. The elastic strands 170, 172, and thus, the continuous length of first and second belt laminates 406, 408 are maintained in a stretched condition while advancing along the machine direction MD.

In some embodiments, the cut line through the elastic laminate 402 created by the cutter 506 may define the inner edge 107b of the first belt laminate 406 and/or the inner edge 109b of the second belt laminate 408. In some embodiments, the first belt laminate 406 and/or the second belt laminate 408 may advance from the cutter 506 to a folding apparatus adapted that folds the cut edges of the first and/or second belt laminates created by the cutter 506. As such, the inner edge 107b of the first belt laminate 406 and/or the inner edge 109b of the second belt laminate 408 may be defined by a fold line extending along the machine direction MD.

It is to be appreciated that the diverter 508 may be configured in various ways. For example, in some embodiments, the diverter 508 may include turn bars angled at 45 degrees or some other angle with respect to the machine direction. In some embodiments, the diverter may include cambered rollers. It is to be appreciated that the first and second belts may be formed by separate continuous lengths of belt material similar to the description above and as such would not require the slitting step or the diverting step. And in some embodiments, the first and second belts may be formed by slitting the outer belt substrate 162 and the inner belt substrate 164 along the machine direction MD before being combined with the elastic material 168.

In some embodiments, the diverter 508 may include a pivot or tracking table, such as for example, the FIFE-500 Web Guiding System, by Maxcess-FIFE Corporation, which can adjust the positions of the continuous length of first and second belt laminates 406, 408 in the cross direction CD. Other suitable pivot or tracking tables are available from Erhardt & Leimer, Inc. The diverter may also include instrumentation and web edge control features that allow for precise active control of the substrate positions.

As previously mentioned, the first belt laminate 406 is separated in the cross direction CD from the second belt laminate 408 to define a gap between the inner longitudinal edge 107b of the first belt laminate 406 and the inner longitudinal edge 109b of the second belt laminate 408. As discussed in more detail below, the first and second belt laminate 406, 408 advance from the diverter 508 to a nip 316 between the carrier apparatus 308 and a roll 318 to be combined with discrete chassis 102.

Referring now to FIGS. 4 and 5C, a continuous length of chassis assemblies 302 are advanced in a machine direction MD and define a width in a cross direction CD. The continuous length of chassis assemblies 302 may include absorbent assemblies 140 sandwiched between topsheet material 138 and backsheet material 136, leg elastics, barrier leg cuffs and the like. As shown in FIG. 5C, portion of the chassis assembly is cut-away to show a portion of the topsheet material 138 and an absorbent assembly 140. The continuous length of chassis assemblies 302 advance to a carrier apparatus 308 and are cut into discrete chassis 102 with knife roll 306, while advancing in the orientation shown in FIG. 5D1, wherein the longitudinal axis 124 of each chassis 102 is generally parallel with the machine direction MD.

After the discrete absorbent chassis 102 are cut by the knife roll 306, the carrier apparatus 308 rotates and advances the discrete chassis 102 in the machine direction MD in the orientation shown in FIG. 5D1. While the chassis 102 shown in FIG. 5D1 is shown with the second laterally extending end edge 146 as a leading edge and the first laterally extending end edge 144 as the trailing edge, it is to be appreciated that in other embodiments, the chassis 102 may be advanced in other orientations. For example, the chassis may be oriented such that the second laterally extending end edge 146 is a trailing edge and the first laterally extending end edge 144 is a leading edge. The carrier apparatus 308 also rotates while at the same time changing the orientation of the advancing chassis 102. In changing the chassis orientation, the carrier apparatus 308 may turn each chassis 102 such that the lateral axis 126 of the chassis 102 is parallel or generally parallel with the machine direction MD, such as shown in FIG. 5D2. The carrier apparatus 308 may also change the speed at which the chassis 102 advances in the machine direction MD to a different speed. FIG. 5D2 shows the orientation of the chassis 102 on the carrier apparatus 308 while advancing in the machine direction MD. More particularly, FIG. 5D2 shows the chassis 102 with the lateral axis 126 of the chassis 102 generally parallel with the machine direction MD, and wherein the second longitudinal side edge 130 is the leading edge and the first longitudinal side edge 128 is the trailing edge. It is to be appreciated that various forms of carrier apparatuses may be used with the methods herein, such as for example, the carrier apparatuses disclosed in U.S. Pat. No. 7,587,966 and U.S. Patent Publication Nos. 2013/0270065 A1; 2013/0270069 A1; 2013/0270066 A1; and 2013/0270067 A1. In some embodiments, the carrier apparatus 308 may rotate at a variable angular velocity that may be changed or adjusted by a controller in order to change the relative placement of the chassis 102 and the advancing belt laminates 406, 408.

As discussed below with reference to FIGS. 4, 5E1, 5E2, 5F, and 5G, the chassis 102 are transferred from the carrier apparatus 308 and combined with advancing, continuous lengths of belt laminates 406, 408, which are subsequently cut to form first and second elastic belts 106, 108 on diapers 100.

As shown in FIGS. 4, 5B, 5E1, and 5E2, the chassis 102 are transferred from the carrier apparatus 308 to a nip 316 between the carrier apparatus 308 and a roll 318 where the chassis 102 is combined with continuous lengths of advancing first belt 406 and second belt 408. The first belt laminate 406 and the second belt laminate material 408 each include a wearer facing surface 312 and an opposing garment facing surface 314. As such, the second surface 162b of the outer layer belt substrate 162 may define some or all the garment facing surface 314, and the second surface 164b of the inner layer belt substrate 164 may define some or all the wearer facing surface 312. The wearer facing surface 312 of the first belt laminate 406 may be combined with the garment facing surface 134 of the chassis 102 along the first waist region 116, and the wearer facing surface 312 of the second belt laminate 408 may be combined with the garment facing surface 134 of the chassis 102 along the second waist region 118. As shown in FIG. 4, adhesive 320 may be intermittently applied to the wearer facing surface 312 of the first and second belt laminates 406, 408 before combining with the discrete chassis 102 at the nip 316 between roll 318 and the carrier apparatus 308.

With reference to FIGS. 4, 5E1, and 5E2 a continuous length of absorbent articles 400 is defined by multiple discrete chassis 102 spaced apart from each other along the machine direction MD and connected with each other by the second belt laminate 408 and the first belt laminate 406. The continuous length of absorbent articles 400 may also be described as a substrate 600 that includes a first surface 602 and an opposing second surface 604. As such, the first surface 602 may correspond with the wearer facing surface 312, and the second surface 604 may correspond with the garment facing surface 314. The substrate 600 also includes a first outer region 606 separated from a second outer region 608 in the cross direction CD by a central region 610. In turn, the first belt laminate 406 may define the first outer region 606, and the second belt laminate 408 may define the second outer region 608. And the multiple discrete chassis 102 and/or absorbent cores 140 that are spaced apart from each other with gaps there between along the machine direction MD may define the central region 610. As such, the first outer region 606 and the second outer region 608 are continuous in the machine direction MD and the central region 610 is discontinuous in the machine direction MD. It is to be appreciated the widths of the regions 606, 608, 610 may vary. For example, in some embodiments, the central region 610 may be about 33% of the width W of the substrate 600. In some embodiments, the first and second outer regions 606, 608 and/or the central region 610 may each be about ⅓ of the width W of the substrate 600.

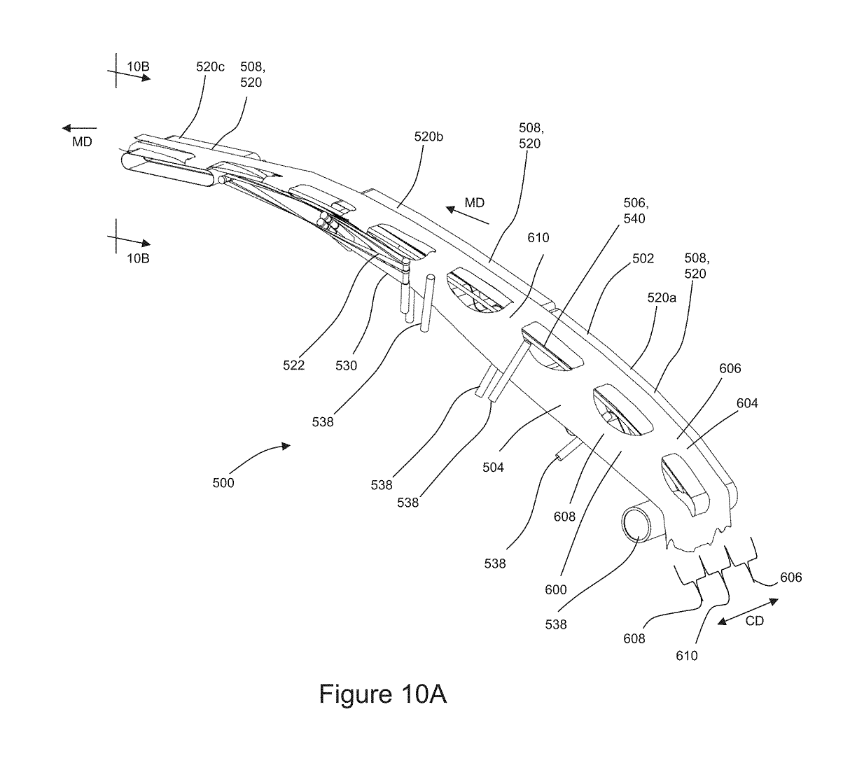

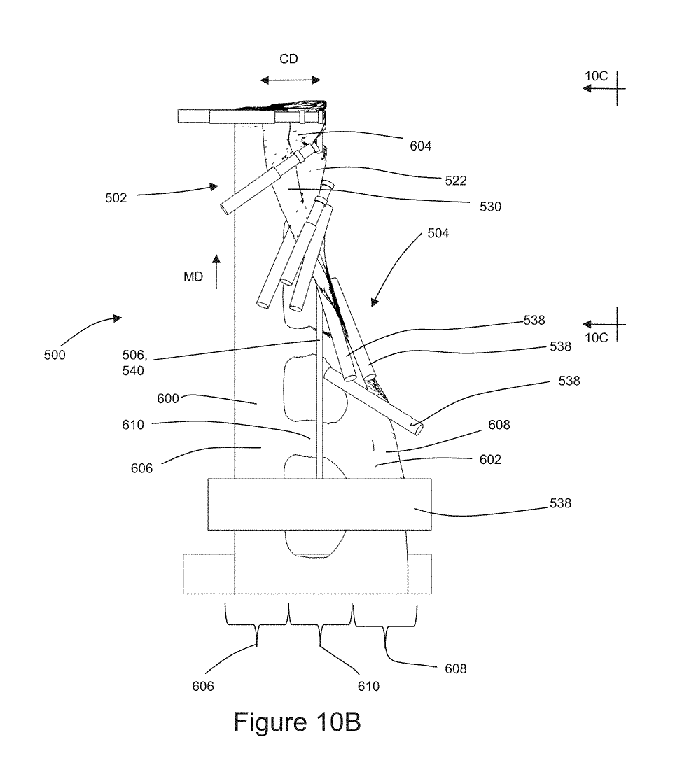

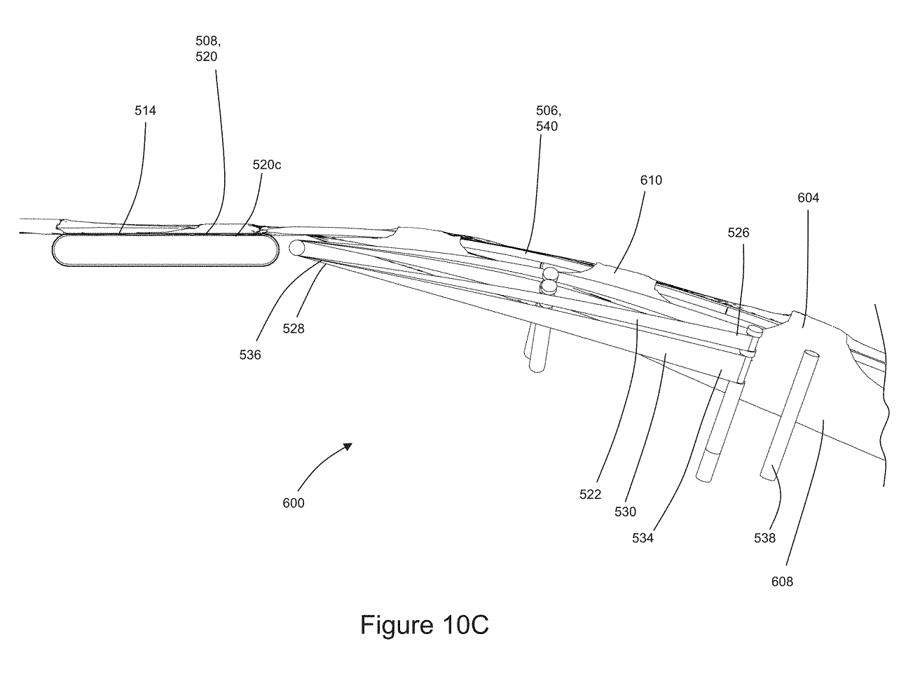

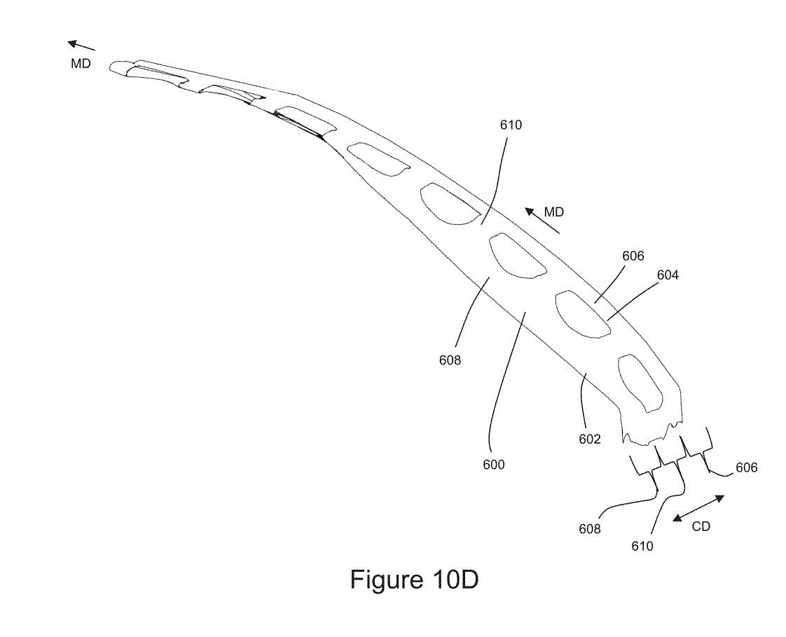

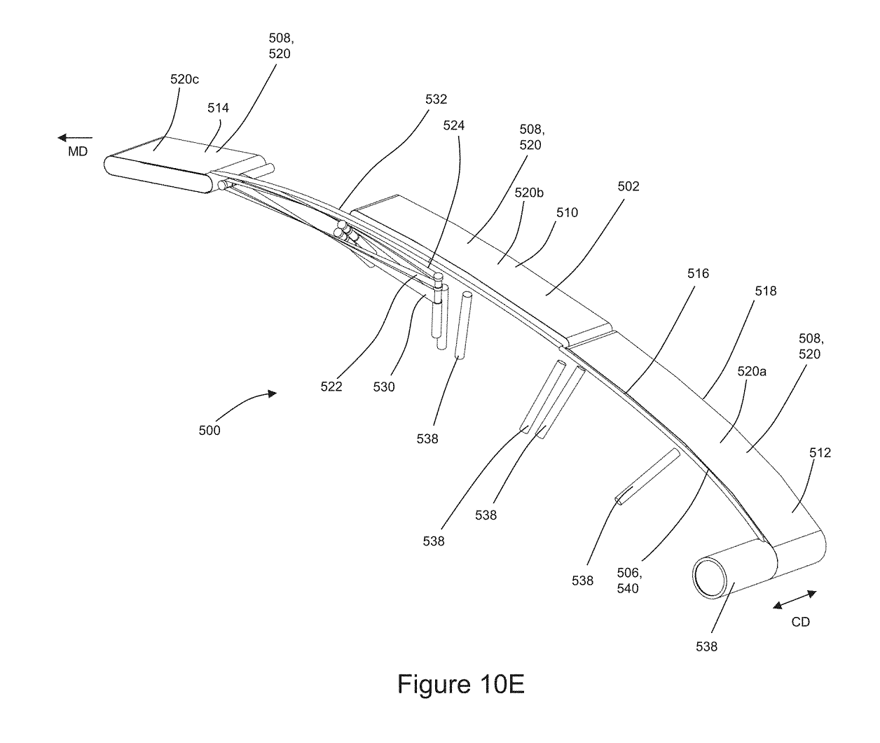

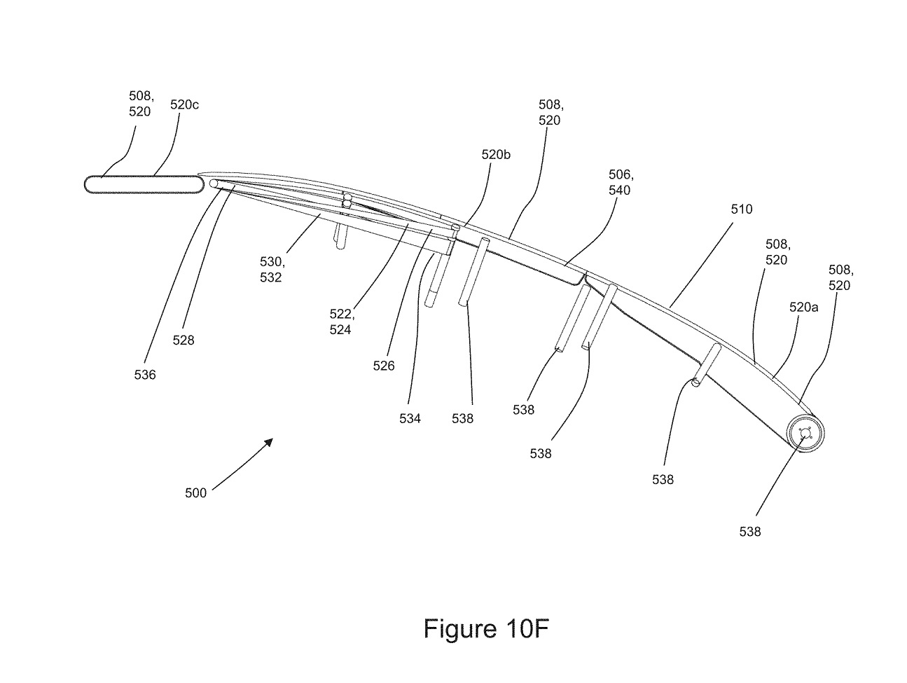

As shown in FIG. 4, the continuous length of absorbent articles 400 advances from the nip 316 to a folding apparatus 500. At the folding apparatus 500, each chassis 102 is folded in the cross direction CD parallel to or along a lateral axis 126 to place the first waist region 116, and specifically, the inner, body facing surface 132 into a facing, surface to surface orientation with the inner, body surface 132 of the second waist region 118. The folding of the chassis also positions the wearer facing surface 312 of the second belt laminate 408 extending between each chassis 102 in a facing relationship with the wearer facing surface 312 of the first belt laminate 406 extending between each chassis 102. Various configurations of the folding apparatus 500 are described in more detail below as folding substrate 600 with reference to FIGS. 10A-12.

As shown in FIGS. 4 and 5F, the folded discrete chassis 102 connected with the first and second belt laminates 406, 408 are advanced from the folding apparatus 500 to a bonder apparatus 334. The bonder apparatus 334 operates to bond an overlap area 362, thus creating discrete bonds 336a, 336b. The overlap area 362 includes a portion of the second belt laminate 408 extending between each chassis 102 and a portion of the first belt laminate 406 extending between each chassis 102. It is to be appreciated that the bonder apparatus 334 may be configured in various ways to create bonds 336a, 336b in various ways, such as for example with heat, adhesives, pressure, and/or ultrasonics. It is also to be appreciated that in some embodiments, the apparatus 300 may also be configured to refastenably bond the overlap area 362, in addition to or as opposed to permanently bonding the overlap area 362. Thus, the discrete bonds 336a, 336b may be configured to be refastenable, such as with hooks and loops.

Referring now to FIGS. 4 and 5G, the continuous length of absorbent articles 400 are advanced from the bonder 334 to a cutting apparatus 338 where the first belt laminate 406 and the second belt laminate 408 are cut along the cross direction CD between adjacent bonds 336a, 336b to create discrete absorbent articles 100. As shown in FIG. 5G, the first belt laminate 406 and the second belt laminate 408 are cut into discrete pieces to form the first and second elastic belts 106, 108, each having a pitch length, PL, extending along the machine direction MD. As such, bond 336a may correspond with and form a first side seam 178 on an absorbent article 100, and the bond 336b may correspond with and form a second side seam 180 on a subsequently advancing absorbent article.

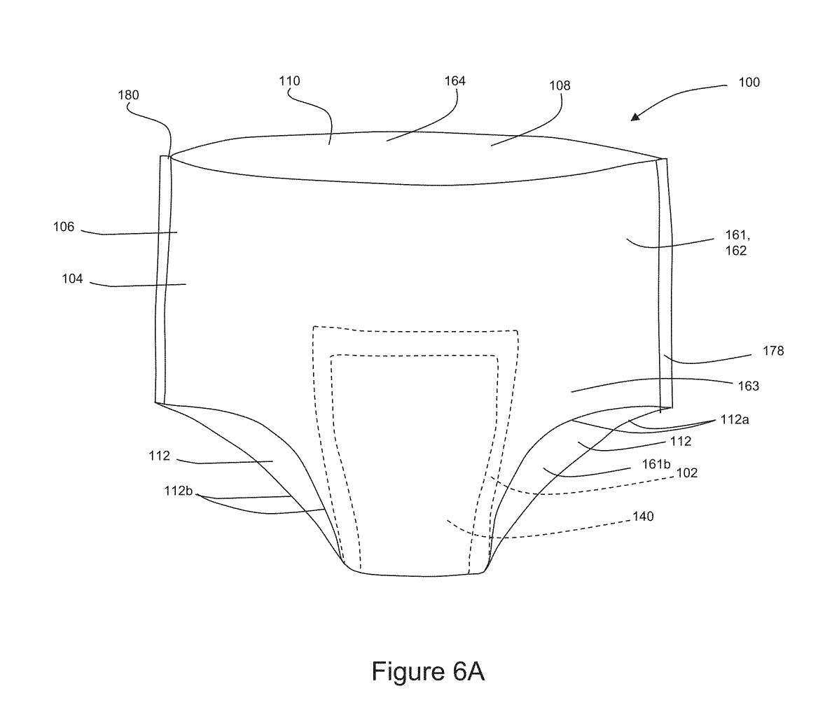

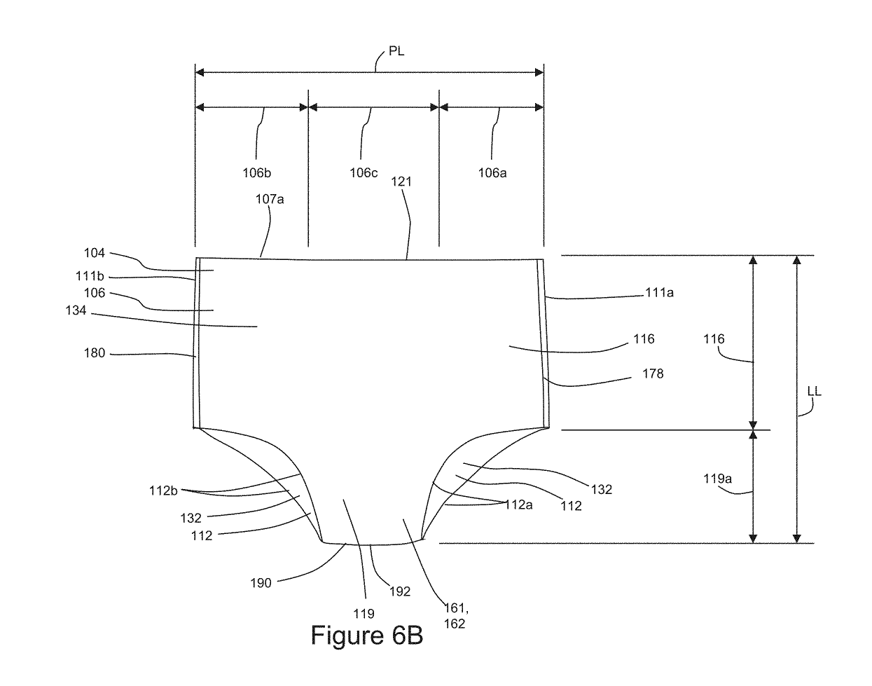

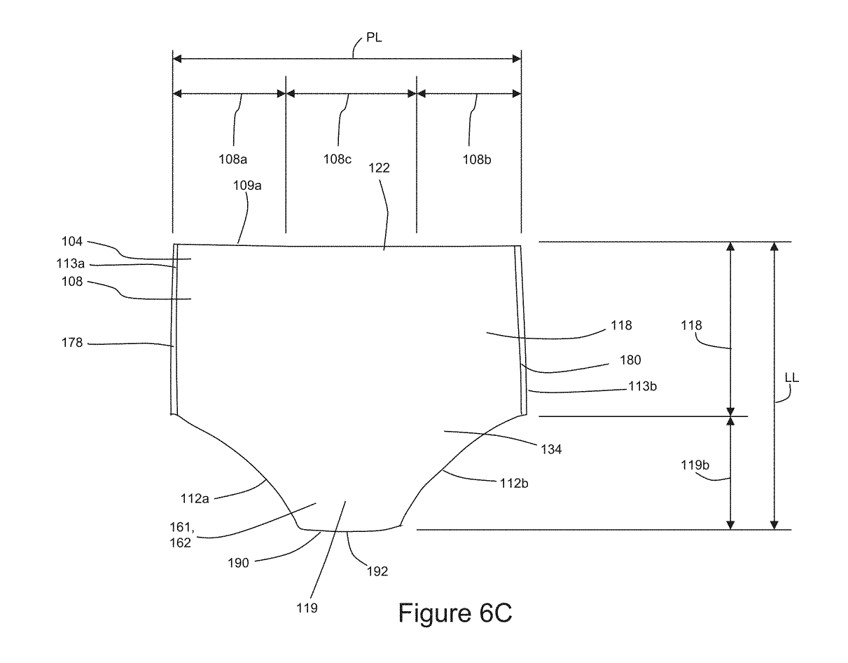

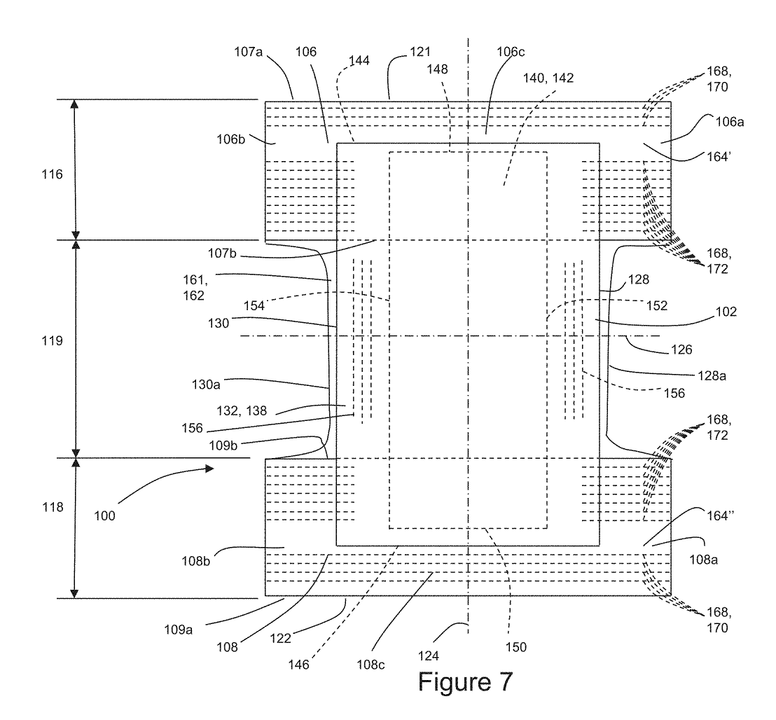

It is to be appreciated that the processes and apparatuses herein may be configured to manufacture various types of diaper pants discussed above. In some embodiments, the diaper pants 100 may include a chassis 102 and elastic belts 106, 108 configured in different ways other than as depicted in FIGS. 1A, 1B, and 2. For example, FIGS. 6A-7 show a diaper pant 100 having many of the same components as described above with reference to FIGS. 1A-2, except the outer layer 162 of the elastic belts 106, 108 is configured as a contiguous outer cover 161 that extends through the first waist region 116, crotch region 119, and second waist region 118. Thus, as shown in FIG. 7, the outer cover 161 also includes a first waist end region 116, a crotch region 119, and an opposing second waist end region 118. The outer cover 161 also includes a garment facing surface 162b and an opposing wearer facing surface 162a. As such, elastic members 168 of the elastic belts 106, 108 may be connected with the wearer facing surface 162a of the outer cover 161. And the chassis 102 may be positioned on the wearer facing surface 162a of the outer cover 161. As such, the backsheet 136 may include a portion of the outer cover 161. In addition, the outer cover 161 may include a first longitudinal side edge 128a and a second longitudinal side edge 130a that are positioned laterally outboard the first longitudinal side edge 128 of the chassis 102 and second longitudinal side edge 130 of the chassis 102, respectively, as shown in FIG. 7. As shown in FIGS. 6A and 7, the first longitudinal side edge 128a may define the perimeter 112a of one leg opening 112, and the second longitudinal side edge 130a may define the perimeter 112b of the other leg opening 112. It is to be appreciated also that the first longitudinal side edge 128a and a second longitudinal side edge 130a may aligned with or positioned laterally inboard of the first longitudinal side edge 128 of the chassis 102 and second longitudinal side edge 130 of the chassis 102, respectively. As such, in some embodiments, the perimeter 112a of one leg opening 112 may be defined by portions of the first longitudinal edges 128, 128a, and the perimeter 112b of the other leg opening may be defined by portions of the second longitudinal edges 130, 130a.

FIG. 6B shows a front plan view of a diaper pant 100 in a laid flat condition illustrating various regions of the diaper pant 100. And 6C shows a rear plan view of the diaper pant 100 in a laid flat condition illustrating various regions of the diaper pant 100. As discussed above, the diaper pant 100 defines include an inner, body facing surface 132, and an outer, garment facing surface 134. The diaper pant 100 also includes a crotch end 190 that is defined by a lateral fold line 192 in the crotch region 119. As such, the lateral fold line 192 divides the crotch region into a first crotch region 119a and a second crotch region 119b.

The diaper pant 100 is shown in FIGS. 6A-6C as having a first elastic belt 106, and a second elastic belt 108. The first belt 106 has a first end region 106a, an opposing second end region 106b, and a central region 106c. And the second belt 108 has a first end region 108a, an opposing second end region 108b, and a central region 108c. The first end regions 106a, 108a are connected together at a first side seam 178, and the second end regions are 106b, 108b are connected together at a second side seam 180. As shown in FIGS. 6B and 6C, the distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108.

The first end region 106a the first belt 106 may extend approximately 20% to 40% of the pitch length PL of the diaper pant 100 in an assembled, laid-flat, relaxed condition, and the first end region 108a the second belt 108 may extend approximately 20% to 40% of the pitch length PL of the diaper pant 100 in an assembled, laid-flat, relaxed condition. The second end region 106b the first belt 106 may extend approximately 20% to 40% of the pitch length PL of the diaper pant 100 in an assembled, laid-flat, relaxed condition, and the second end region 108b the second belt 108 may extend approximately 20% to 40% of the pitch length of the diaper pant 100 in an assembled, laid-flat, relaxed condition. The central region 106c the first belt 106 may extend approximately 20% to 60% of the pitch length PL of the diaper pant 100 in an assembled, laid-flat, relaxed condition, and the central region 108c the second belt 108 may extend approximately 20% to 60% of the pitch length PL of the diaper pant 100 in an assembled, laid-flat, relaxed condition.

The diaper pant 100 in FIGS. 6B and 6C is also shown as having a longitudinal length LL that is defined by the distance between the first waist edge 121 and the crotch end 190 (or the lateral fold line 192), or if longer, the distance from the second waist edge 122 to the crotch end 190 (or the lateral fold line 192). The longitudinal length LL may be measured along the longitudinal centerline 124 of the diaper pant 100. As shown in FIGS. 6B-6C, the first waist region 116 extends a distance generally in the longitudinal direction from the waist edge 121 along the side seams 178, 180 to the leg openings 112, and the second waist region 118 extends a distance generally in the longitudinal direction from the waist edge 122 along the side seams 178, 180 to the leg openings 112. Hence, a first crotch region 119a extends a distance from the crotch end 190 to the first waist region 116, and a second crotch region 119b extends a distance from the crotch end 190 to the second waist region 118. In some embodiments, the first waist region 116 and/or the second waist region 118 may extend about two-thirds the longitudinal length LL of the assembled diaper pant 100. In addition, the first crotch region 119a and/or the second crotch region 119b may extend about one-third the longitudinal length LL of the assembled diaper pant 100.

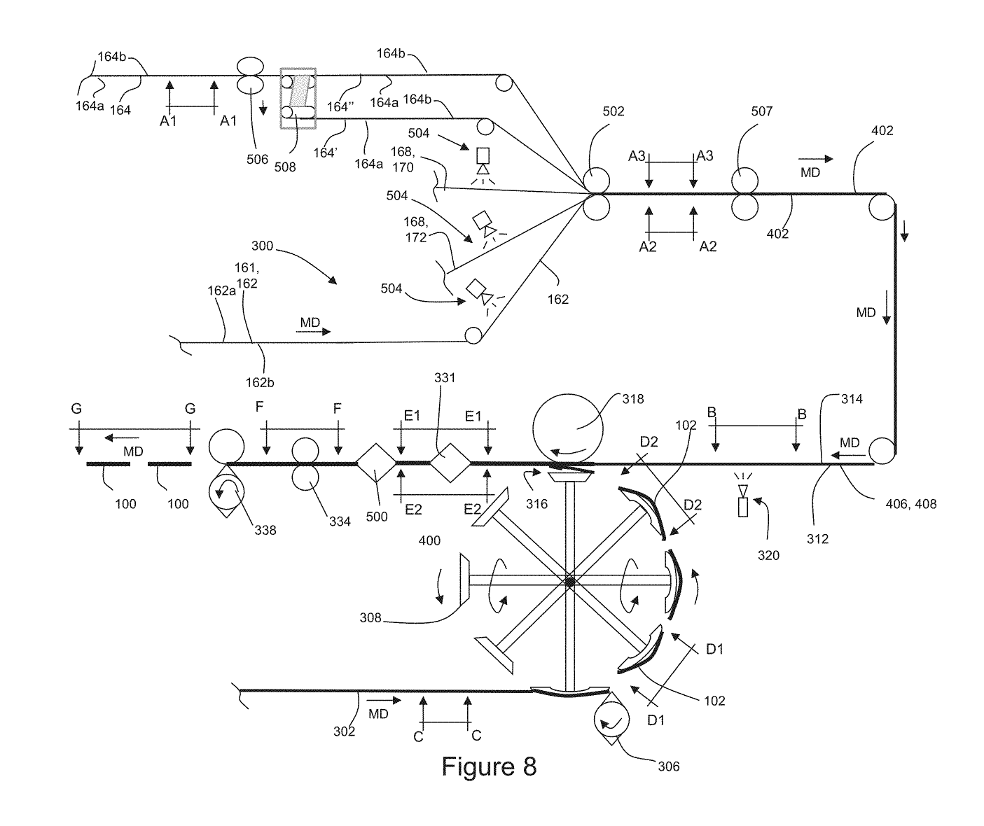

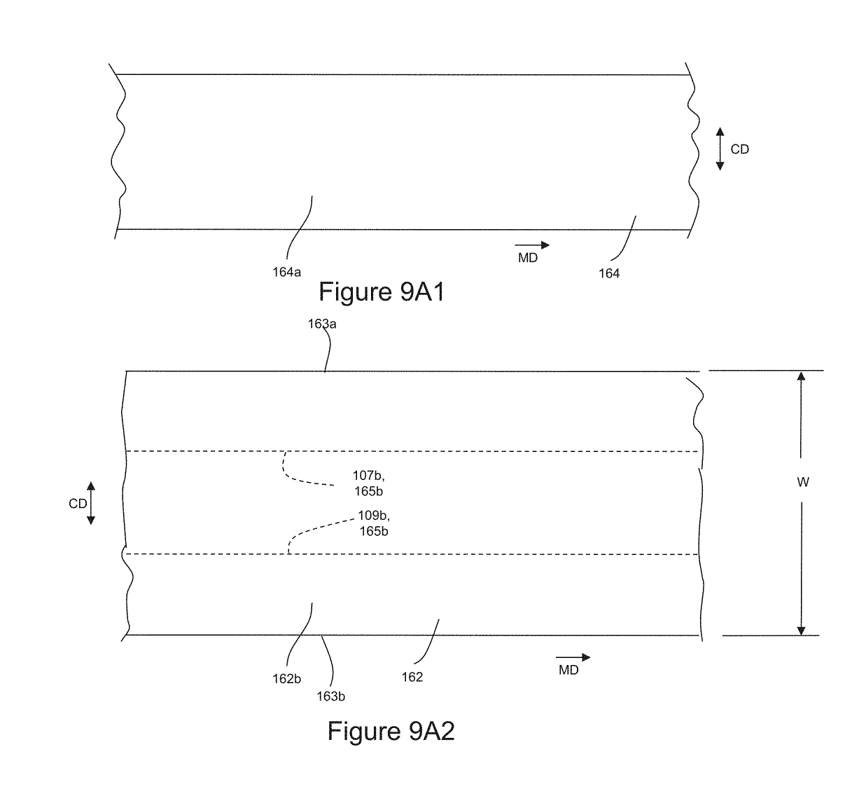

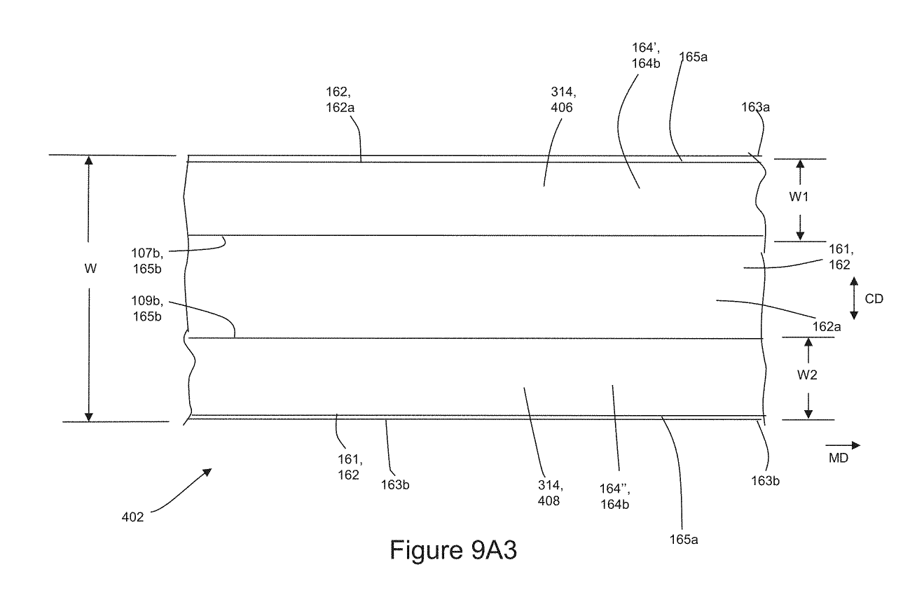

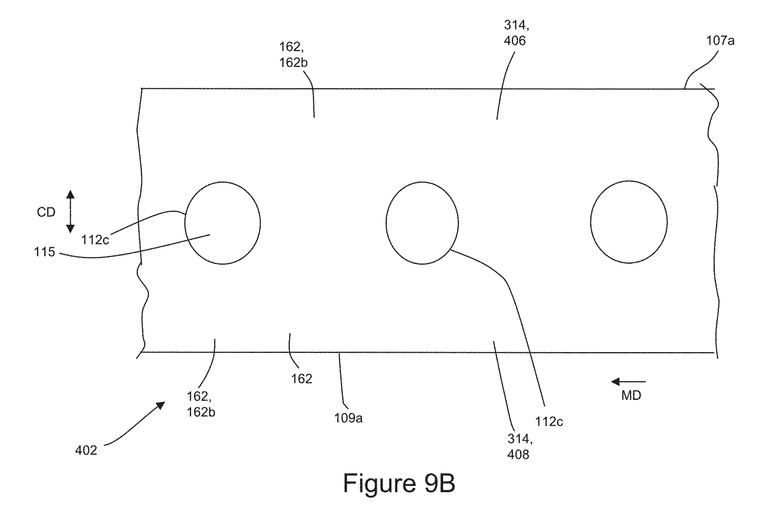

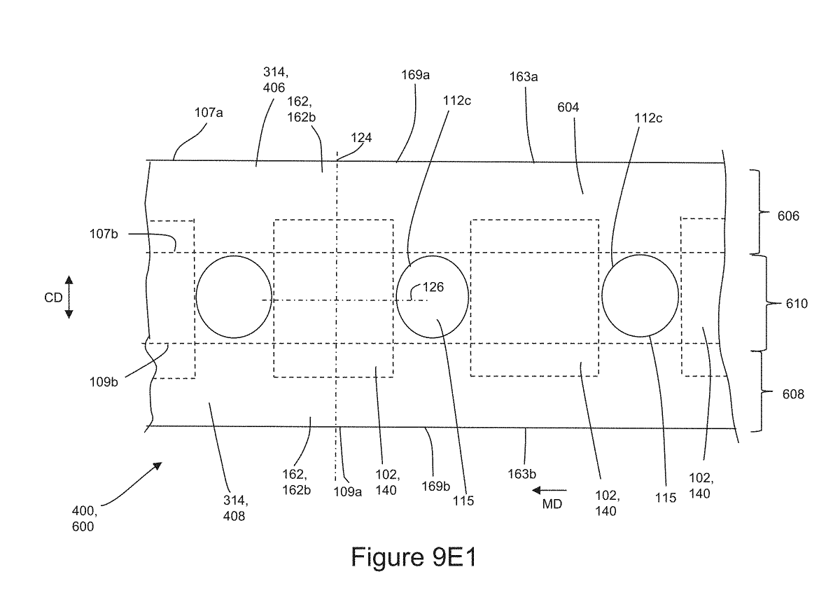

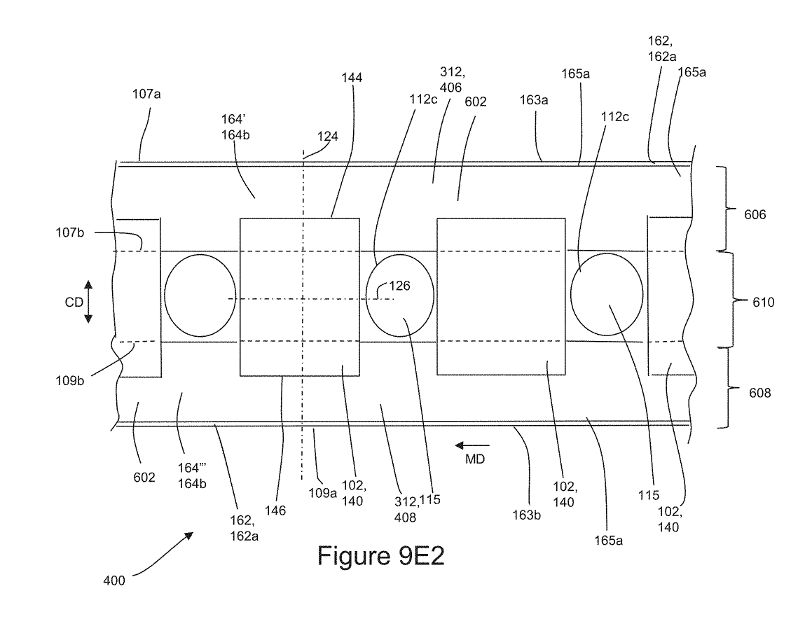

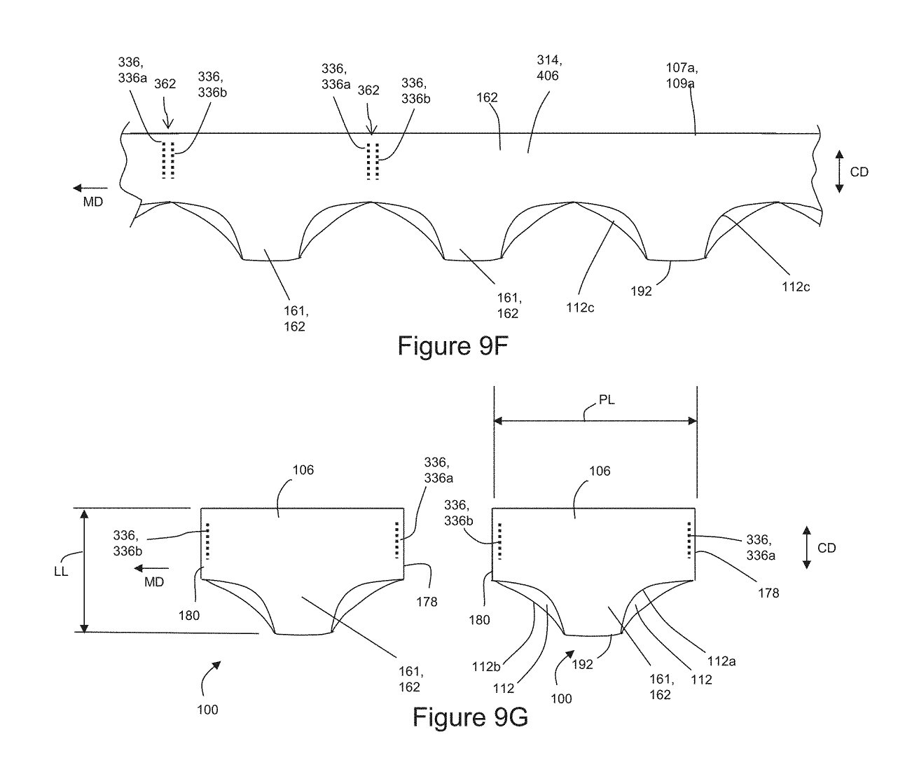

FIG. 8 shows a converting apparatus 300 configured to assemble diaper pants such as shown in FIGS. 6A-7. As shown in FIG. 8, a first continuous substrate layer in the form of a continuous length of outer layer belt substrate 162 is combined with first and second separate continuous lengths of inner layer belt substrates 164′, 164″ and elastics 168 form a continuous elastic laminate 402. The outer layer belt substrate 162 also defines the outer cover 161 discussed above with reference to FIGS. 6A-7. With reference to FIGS. 8, 9A1, 9A2, and 9B, continuous lengths of outer layer belt substrate 162, first and second inner layers of belt substrate 164′, 164″, outer elastic strands 170 and inner elastic strands 172 are advanced in a machine direction MD and combined at nip rolls 502 to form the continuous elastic laminate 402.

With reference to FIG. 8, before entering the nip rolls 502, a continuous inner belt substrate 164 having a first surface 164a and an opposing second surface 164b advances in the machine direction MD to a cutter 506 that cuts the continuous inner belt substrate 164 into inner layer belt substrates 164′, 164″.

With continued reference to FIG. 8, the cutter 506 operates to cut the inner belt substrate 164 along the machine direction to form the inner layer belt substrates 164′, 164″. As discussed above, the cutter 506 may be configured in various ways. For example, in some embodiments the cutter 506 may be a slitter or a die cutter that separates the inner belt substrate into two continuous belt substrates with either a straight line cut and/or a curved line cut. The cutter 506 may also be configured as a perforator that perforates the belt material with a line of weakness and wherein the belt material is separated along the line of weakness in a later step. From the cutter 506, the inner layer belt substrates 164′, 164′ advance through a diverter 508 that separates the inner layer belt substrates 164′, 164″ from each other in the cross direction CD.

As shown in FIG. 8, before entering the nip rolls 502, the outer elastic strands 170 and inner elastic strands 172 are stretched in the machine direction MD. In addition, adhesive 504 may applied to the elastic strands 170, 172 as well as either or both of the continuous lengths of outer layer belt substrate 162 and inner layer belt substrates 164′, 164″ before entering nip rolls 502. As such, the elastic strands 168 are bonded between the first surface 162a of the outer layer belt substrate 162 and the first surfaces 164a of inner layer belt substrates 164′, 164″ at the nip rolls 502. Further, adhesive 504 may be applied intermittently along the lengths of the inner elastic strands 172 and/or intermittently along the length of either or both of the continuous lengths of outer layer belt substrate 162 and inner layer belt substrates 164′, 164″ before entering nip rolls 502. As previously discussed, the inner elastic strands 172 may be intermittently bonded to either or both of the continuous lengths of outer layer belt substrate 162 and inner layer belt substrates 164′, 164″ along the machine direction MD.

As shown in FIGS. 8, 9A2, and 9A3, the outer belt substrate 162 includes first surface 162a and an opposing second surface 162b, and defines a width W in the cross direction between opposing longitudinal edges 163a, 163b. The first inner belt substrate 164′ includes first surface 164a and an opposing second surface 164b, and defines a width in the cross direction CD between opposing first and second longitudinal edges 165a, 165b. And the second inner belt substrate 164″ includes first surface 164a and an opposing second surface 164b, and defines a width in the cross direction CD between opposing first and second longitudinal edges 165a, 165b. As shown in FIG. 9A2, the width W of the outer belt substrate 162 may be greater than the widths of the inner belt substrates 164′, 164″. And the width W of the outer belt substrate 162 may also define the width W of the elastic laminate 402.

As shown in FIGS. 8 and 9A3, the continuous elastic laminate 402 includes a first elastic belt laminate 406 and a second elastic belt laminate 408. More particularly, the combination of the outer layer belt substrate 162, the first inner layer of belt substrate 164′, and elastic strands 168 defines the first belt laminate 406. And the combination of the outer layer belt substrate 162, the second inner layer of belt substrate 164″, and elastic strands 168 defines the second belt laminate 408. The first belt laminate 406 includes an outer longitudinal edge 163a and an inner longitudinal edge 107b that may define a substantially constant width, W1, in the cross direction CD. The inner longitudinal edge 107b may be defined by the second longitudinal edge 165b of the first inner belt substrate 164′. The second belt laminate 408 includes an outer longitudinal edge 163b and an inner longitudinal edge 109b that may define a substantially constant width, W2, in the cross direction CD. The inner longitudinal edge 109b may be defined by the second longitudinal edge 165b of the second inner belt substrate 164″. In some configurations, W2 may be equal to W1. It is also to be appreciated that in some configurations, W1 may be less than or greater than W2. The first belt laminate 406 is separated in the cross direction from the second belt laminate 408 to define a gap between the inner longitudinal edge 107b of the first belt laminate 406 and the inner longitudinal edge 109b of the second belt laminate 408.

With continued reference to FIG. 8, from the nip rolls 502 the continuous elastic laminate 402 advances in the machine direction MD to a cutter 507 that removes material from a central region of the continuous elastic laminate 402 to form holes 115 defined by perimeter edges 112c, such as shown in FIG. 9B. The holes 115 are discrete and may be spaced apart from each other along the machine direction MD. The perimeter edges 112c may define all or portions of the perimeters 112a, 112b of the leg openings 112 mentioned above and shown in FIG. 6A.

It is to be appreciated that the cutter 507 may be configured to remove material from only the outer layer belt substrate 162. In some configurations, the cutter 507 may be configured to remove material from the outer belt substrate 162 as well as the first inner layer belt substrate 164′ and/or second inner layer belt substrate 164″. The cutter 507 may also be configured as a perforator that perforates the belt material with a line of weakness and wherein the belt material is separated along the line of weakness in a later step. It is also to be appreciated that the cutter 507 may be configured to form holes 115 in the continuous elastic laminate 402 before or after the continuous elastic laminate 402 is combined with the chassis 102.

As discussed above with reference to FIGS. 4, 5C, 5D1, and 5D2, and as shown in FIG. 8, a continuous length of chassis assemblies 302 are advanced in a machine direction MD to a carrier apparatus 308 and are cut into discrete chassis 102 with knife roll 306, while advancing in the orientation shown in FIG. 5D1. After the discrete absorbent chassis 102 are cut by the knife roll 306, the carrier apparatus 308 rotates and advances the discrete chassis 102 in the machine direction MD in the orientation shown in FIG. 5D1. The carrier apparatus 308 also rotates while at the same time changing the orientation of the advancing chassis 102. In changing the chassis orientation, the carrier apparatus 308 may turn each chassis 102 such that the lateral axis 126 of the chassis 102 is parallel or generally parallel with the machine direction MD, such as shown in FIG. 5D2.

As shown in FIGS. 8, 9E1, and 9E2, the chassis 102 are transferred from the carrier apparatus 308 to a nip 316 between the carrier apparatus 308 and a roll 318 where the chassis 102 is combined with the continuous elastic laminate 402. The chassis 102 may be spaced apart from each other along the machine direction MD on the continuous elastic laminate 402, wherein at least one hole 115 is positioned between two consecutive chassis 102. The continuous elastic laminate 402 includes a wearer facing surface 312 and an opposing garment facing surface 314. As such, the second surface 162b of the outer layer belt substrate 162 may define the garment facing surface 314. And the first surface 162a of the outer layer belt substrate 162 and the second surfaces 164b of the inner layer belt substrates 164′, 164″ may define the wearer facing surface 312. The wearer facing surface 312 of the continuous elastic laminate 402 may be combined with the garment facing surface 134 of the chassis 102. As shown in FIG. 8, adhesive 320 may be intermittently applied to the wearer facing surface 312 of the continuous elastic laminate 402 before combining with the discrete chassis 102 at the nip 316 between roll 318 and the carrier apparatus 308.

With continued reference to FIGS. 8, 9E1, and 9E2, a continuous length of absorbent articles 400 is defined by multiple discrete chassis 102 spaced apart from each other, separated by the holes 115 along the machine direction MD, and connected with each other by the continuous elastic laminate 402. The continuous length of absorbent articles 400 may also be described as a substrate 600 that includes a first surface 602 and an opposing second surface 604. As such, the first surface 602 may correspond with the wearer facing surface 312, and the second surface 604 may correspond with the garment facing surface 314. The substrate 600 also includes a first outer region 606 separated from a second outer region 608 in the cross direction CD by a central region 610. In turn, the first belt laminate 406 may define the first outer region 606, and the second belt laminate 408 may define the second outer region 608. And the multiple discrete chassis 102 and/or absorbent cores 140 that are spaced apart from each other with holes 115 there between along the machine direction MD may define the central region 610. As such, the first outer region 606 and the second outer region 608 are continuous in the machine direction MD and the central region 610 is discontinuous in the machine direction MD. It is to be appreciated the widths of the regions 606, 608, 610 may vary. For example, in some embodiments, the central region 610 may be about 33% of the width W of the substrate 600. In some embodiments, the first and second outer regions 606, 608 and/or the central region 610 may each be about ⅓ of the width W of the substrate 600.

As shown in FIG. 8, the continuous length of absorbent articles 400 advances from the nip 316 to a folding apparatus 500. At the folding apparatus 500, the continuous elastic laminate 402 and each chassis 102 are folded in the cross direction CD parallel to or along a lateral axis 126 to place the first waist region 116, and specifically, the inner, body facing surface 132 into a facing, surface to surface orientation with the inner, body surface 132 of the second waist region 118. The folding operation creates the lateral fold line 192 that defines the crotch end 190 discussed above with reference to FIGS. 6B and 6C. The folding of the chassis also positions the wearer facing surface 312 of the second belt laminate 408 extending between each chassis 102 in a facing relationship with the wearer facing surface 312 of the first belt laminate 406 extending between each chassis 102. Various configurations of the folding apparatus 500 are described in more detail below as folding substrate 600 with reference to FIGS. 10A-12.

As shown in FIGS. 8 and 9F, the folded continuous length of absorbent articles 400 are advanced from the folding apparatus 500 to a bonder apparatus 334. The bonder apparatus 334 operates to bond an overlap area 362, thus creating discrete bonds 336a, 336b. The overlap area 362 includes a portion of the second belt laminate 408 extending between each chassis 102 and a portion of the first belt laminate 406 extending between each chassis 102. It is to be appreciated that the bonder apparatus 334 may be configured in various ways to create bonds 336a, 336b in various ways, such as for example with heat, adhesives, pressure, and/or ultrasonics. It is also to be appreciated that in some embodiments, the apparatus 300 may be configured to refastenably bond the overlap area 362, in addition to or as opposed to permanently bonding the overlap area 362. Thus, the discrete bonds 336a, 336b may be configured to be refastenable, such as with hooks and loops.

Referring now to FIGS. 8 and 9G, the continuous length of absorbent articles 400 are advanced from the bonder 334 to a cutting apparatus 338 where the first belt laminate 406 and the second belt laminate 408 are cut along the cross direction CD between adjacent bonds 336a, 336b to create discrete absorbent articles 100. As shown in FIG. 9G, the continuous length of absorbent articles 400 are cut into discrete pieces to form the first and second elastic belts 106, 108, each having a pitch length, PL, extending along the machine direction MD and longitudinal length LL extending in the cross direction CD. As such, bond 336a may correspond with and form a first side seam 178 on an absorbent article 100, and the bond 336b may correspond with and form a second side seam 180 on a subsequently advancing absorbent article.

As previously mentioned with reference to FIGS. 4 and 8, the converting apparatuses 300 herein may include a folding apparatus 500 to fold the continuous length of absorbent articles 400 in the cross direction CD. In addition, the continuous length of absorbent articles 400 discussed above with reference to FIGS. 5E1, 5E2, 9E1, and 9E2 may be described generally as a substrate 600 including a first surface 602 and an opposing second surface 604. The substrate further includes a first outer region 606 separated from a second outer region 608 in the cross direction CD by a central region 610, wherein the first outer region 606 and the second outer region 608 are continuous in the machine direction MD and the central region 610 is discontinuous in the machine direction MD. As discussed in more detail below, the folding apparatus 500 is configured to mitigate the undesirable results of frictional forces acting on the central region 610 during the folding process, such as discussed above with reference to Figures A-D.

For example, FIGS. 10A-10D show an isometric view of a substrate 600 advancing in a machine direction MD along an embodiment of a folding apparatus 500 that folds the substrate 600 to position the first surface 602 of the second outer region 608 in a facing relationship with the first surface 602 of the first outer region 606. More particularly, the folding apparatus 500 includes a first advancement path 502 and a second advancement path 504 extending the machine direction MD. The first and second advancement paths 502, 504 are configured such that the first outer region 606 of the substrate 600 is conveyed along the first advancement path 502 and the second outer region 608 is conveyed along the second advancement path 504 while the central region 610 is folded along and around a folding axis 506. As the central region 610 is folded around the folding axis 506, the first surface 602 of the second outer region 608 brought into a facing relationship with the first surface 602 of the first outer region 606. Although the second outer region 608 is depicted as being folded or displaced 180° around the folding axis 506 during the folding operation, it is to be appreciated that the folding apparatus 500 may be configured such that either or both the first outer region 606 and the second outer region 608 may be folded or displaced around the folding axis 506 during the folding operation. For example, the folding apparatus 500 may be configured such that both the first outer region 606 and the second outer region 608 are displaced or folded about 90° around the folding axis 506 during the folding operation.

As shown in FIGS. 10A, 10E, and 10F, the first advancement path 502 of the folding apparatus 500 may include a first conveyor 508 with a carrier surface 510 extending in the machine direction MD from an upstream end region 512 to a downstream end region 514. The first conveyor 508 also includes a first longitudinal edge 516 separated from a second longitudinal edge 518 in the cross direction CD. The first conveyor 508 may be configured to include a conveyor belt 520 that conveys the first outer region 606 of the substrate 600 in the machine direction MD during the folding process. It is to be appreciated that the first conveyor may be configured to include one or more conveyor belts 520. Although the first conveyor 508 is depicted as including three separate conveyor belts 520a, 520b, 520c arranged in series along the machine direction MD, it is to be appreciated that various type of conveyor arrangements may be used. For example, the first conveyor 508 may be configured to include one or more conveyor belts arranged in the machine direction MD and/or cross direction CD along the first advancement path 502. In some configurations, the first conveyor 508 may include one or more endless belts supported by a plurality of rollers. In addition, the first conveyor 508 may be connected with a vacuum source to apply vacuum pressure to the substrate 600. As such, the first conveyor may be configured apply vacuum pressure to the substrate 600, in turn, vacuum forces exerted on the substrate may help to hold the first outer region 606 and/or central region 610 of the substrate 600 in position on the carrier surface 510 of the first conveyor 508 and may help prevent the substrate 600 from slipping on the carrier surface 510 during the folding operation.

With reference to FIGS. 10A-10C, 10E, and 10F, the second advancement path of the folding apparatus 500 may include a second conveyor 522 with a carrier surface 524 extending in the machine direction MD from an upstream end region 526 to a downstream end region 528. In particular, the upstream end region 526 is angularly offset from the downstream end region 528 to define a generally helical-shaped conveyance path along the machine direction MD. The second advancement path 504 of the folding apparatus 500 may also include a third conveyor 530 with a carrier surface 532 extending in the machine direction MD from an upstream end region 534 to a downstream end region 536, wherein the upstream end region 534 is angularly offset from the downstream end region 536 to define a generally helical-shaped conveyance path along the machine direction MD. As discussed in more detail below, the second conveyor 522 may contact and convey the second surface 604 of the central region 610 of the substrate 600 in the machine direction MD during the folding operation. In addition, the third conveyor 530 may contact and convey the second surface 604 of the second outer region 608 of the substrate 600 in the machine direction MD during the folding operation. The helical shaped conveyance paths of the second and third conveyors 522, 530 guide the second outer region 608 of the substrate 600 around the folding axis 506 during the folding operation.

The second conveyor 522 and/or the third conveyor 530 may also be connected with one or more vacuum sources to apply a vacuum pressure to the substrate 600 while conveying the substrate 600 during the folding operation. As discussed below, applying vacuum pressure to the substrate 600 helps to hold the substrate in position on the carrier surfaces 524, 532 of the second conveyor 522 and/or third conveyor 530 and helps to prevent the substrate 600 from slipping on the carrier surfaces. In turn, the vacuum forces may help overcome frictional forces that may act on the substrate 600 during the folding operation. As previously discussed, such frictional forces acting on the substrate 600 during the folding operation may cause the shapes of folded products, such as diapers, to become askew and/or asymmetrical. Thus, the application of vacuum forces to the substrate 600 by the second conveyor 522 and/or third conveyor 530 during the folding operation may help mitigate the effects of the frictional forces that result in misshaped products.

With reference to FIGS. 10A-10C, 10E, and 10F, the second advancement path 504 of the folding apparatus 500 may also include a plurality of rollers 538 positioned upstream of the second conveyor 522 and third conveyor 530. As discussed in more detail below, the rollers 538 may contact, guide, and/or convey the first surface 602 and/or the second surface 604 of the second outer region 608 and/or the central region 610 of the substrate 600 in the machine direction MD during the folding operation. Each roller 538 may have different angular orientations relative to another to provide a generally helical-shaped conveyance path along the machine direction MD, such as described for example in U.S. Patent Publication No. 2013/0203580 A1, which is incorporated by reference herein. It is to be appreciated that the folding apparatus 500 may include various other arrangements of components than those described and shown in herein. For example, some configurations of the folding apparatus 500 may include a twisted belt conveyor arrangement in combination with or instead of a plurality of rollers 538.

As previously mentioned, during the folding operation, the central region 610 of the substrate 600 is folded around the folding axis 506 while the first outer region 606 and the second outer region 608 advance in the machine direction MD. The folding axis 506 extends in the machine direction MD and may be straight and/or curved, such as disclosed for example in U.S. Patent Publication No. 2013/0203580 A1. For example, the folding axis 506 may be defined by an arc extending in the machine direction MD, wherein second outer region 608 of the substrate 600 is helically folded or displaced toward the inside of the arc. In addition, such a curved or arc-shaped folding axis 506 may be configured such the first outer region 606 and the second outer region 608 of the substrate 600 advance along web paths having substantially equal lengths during the folding process. It is to be appreciated that the folding axis 506 may be defined in various ways and may extend in various lengths along the machine direction MD. In some configurations, the folding axis 506 may be defined by a continuous arc. In other configurations, the folding axis 506 may be defined by a plurality of arcs of different radii. In yet other embodiments, the folding axis 506 may be defined by one or more arcs in combination with one or more straight portions. In some embodiments, the folding axis 506 may include straight segments connected with arcuate segments. In some embodiments, such straight segments may approximate a chord of the ideal folding arc between successive folding segments.

With reference to FIGS. 10A, 10B, 10E, and 10F, the folding apparatus may include a rail 540 that defines all or a portion of the folding axis 506. In turn, the rail 540 may be curved and/or straight and may extend for a length in the machine direction MD. As shown in FIGS. 10B and 10E, the rail 540 may be located in a cross directional position between the first advancement path 502 and the second advancement path 504 extending the machine direction MD. In some configurations, the rail 540 may extend for a length along the first longitudinal edge 516 of the first conveyor 508 and adjacent the second conveyor 522 as well as some or all the rollers 538.

With reference to FIGS. 10A-10F, the substrate 600 may advance to the folding apparatus 500 in a substantially flat configuration. During the folding operation, the first outer region 606 is conveyed in the machine direction MD along the first advancement path 502 by the first conveyor 508, wherein the carrier surface 510 contacts the first surface 602 of the first outer region 606 of the substrate 600. At the same time, the second outer region 608 advances along the second advancement path 504 in the machine direction MD. During advancement, the second outer region 608 is initially folded or displaced by the rollers 538, which in turn, wraps the central region 610 partially around the rail 540. More particularly, the first surface 602 of the central region 610 of the substrate 600 is partially wrapped around the rail 540. It is be appreciated that the rollers 538 may be arranged to contact the first surface 602 and/or second surface 604 of the second outer region 608 of the substrate 600.

Downstream of the rollers 538, the substrate 600 may be conveyed by the second conveyor 522 and/or the third conveyor 530. In particular, the carrier surface 524 of the second conveyor 522 contacts the second surface 604 of the central region 610 while first surface 602 of the partially wrapped central region 610 advances along the length of the rail 540. In addition, the carrier surface 524 of the third conveyor 530 contacts the second surface 604 of the second outer region 608. It is to be appreciated that the folding apparatus 500 may be configured such that the carrier surface 524 of the second conveyor 522 contacts only the second surface 604 of the central region 610 or both second surface 604 of the central region 610 and the second outer region 608. It is also to be appreciated that the folding apparatus 500 may be configured such that the carrier surface 532 of the third conveyor 530 contacts only the second surface 604 of the second outer region 608 or both second surface 604 of the second outer region 608 and the central region 610. As the substrate 600 is conveyed from the upstream end regions 526, 534 to the downstream end regions 528, 536 of the second and third conveyors 522, 530, the second outer region 608 is further folded or displaced by the generally helical-shaped travel paths of the second and third conveyors 522, 534. More particularly, the second and third conveyors 522, 530 convey and guide the second outer region 608 around the rail 540 to position the first surface 602 of the second outer region 608 in a facing relationship with the first surface 602 of the first outer region 606, which in turn, further wraps the first surface 602 of the central region 610 of the substrate 600 around the rail 540.

As previously discussed, during the folding process as the substrate 600 advances in the machine direction MD, the first surface 602 of the central region 610 makes contact with and wraps around the rail 540. In turn, as the central region 610 advances along the rail 540, frictional forces between the first surface 602 of the substrate 600 and the rail 540 act on central region 610 of the substrate 600 in a direction that is opposite of the machine direction MD and upstream relative to the advancing first outer region 606 and second outer region 608. To help mitigate the effects of the frictional forces, the first conveyor 508, the second conveyor 522, and/or the third conveyor 530 may apply vacuum forces to the substrate 600 to help hold the second surface 604 of the substrate 600 against the carrier surfaces 510, 524, 532. In turn, the vacuum pressures may help the first conveyor 508, the second conveyor 522, and/or the third conveyor 530 pull the substrate 600 in the machine direction MD along the rail 540, while overcoming the frictional forces and helping to mitigate deformations in the folded substrate caused by such frictional forces.

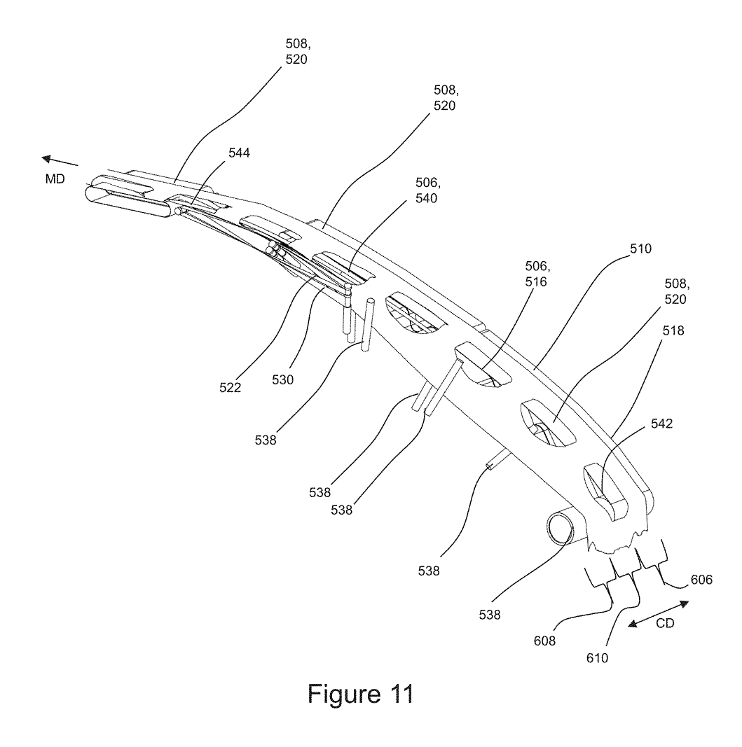

It is to be appreciated that the folding apparatus 500 may be configured in additional ways to further reduce or mitigate the effects of frictional forces caused by the central region 610 of the substrate 600 advancing along the rail 540. In some configurations, the rail 540 may be relatively short. For example, the folding apparatus 500 in FIG. 11 includes a rail 540 that extends for a length that is less than the lengths of the first and/or second advancement paths 502, 504. As shown in FIG. 11, an upstream portion 542 of the folding axis 506 is defined by the first longitudinal edge 516 of the first conveyor 508 and a downstream portion 544 of the folding axis 506 is defined by the rail 540. With reference to FIG. 11, the substrate 600 may advance to the folding apparatus 500 in a substantially flat configuration. During the folding operation, the first outer region 606 of the substrate 600 is conveyed in the machine direction MD along the first advancement path 502 by the first conveyor 508, wherein the carrier surface 510 contacts the first surface 602 of the first outer region 606 of the substrate 600. At the same time, the second outer region 608 advances along the second advancement path 504 in the machine direction MD. During advancement, the second outer region 608 is initially folded or displaced by the rollers 538, which in turn, partially fold the central region 610 along the first longitudinal edge 516 of the first conveyor 508. The partially folded central region 610 of the substrate 600 also advances onto the rail 540. Downstream of the rollers 538, the substrate 600 may be conveyed by the second conveyor 522 and/or the third conveyor 530 to continue folding the substrate 600 around the rail 540 to place the second outer region 608 in a facing relationship with the first outer region 606 as discussed above.

It is to be appreciated that the partially folded central region 610 of the substrate 600 may advance onto the rail 540 at various locations or times during the folding process depending on the length and/or machine direction position of the rail 540 relative the first conveyor 508, rollers 538, second conveyor 522, and/or third conveyor 530. For example, the partially folded central region 610 may advance onto the rail 540 at locations upstream or downstream of where the second and/or third conveyors 522, 530 begin to contact the substrate 600. In some configurations, partially folded central region 610 may advance onto the rail 540 at a location where the second and/or third conveyors 522, 530 begin to contact the substrate 600. In some examples, the folding apparatus 500 may be configured to advance the central region 610 onto the rail 540 depending on a relative angle between the first outer region 606 of the substrate 600 and the second outer region 608. For example, the substrate may be partially folded such that the second outer region 608 is separated from first outer region 606 by an angle of about 90 degrees when the central region 610 advances onto the rail 540.

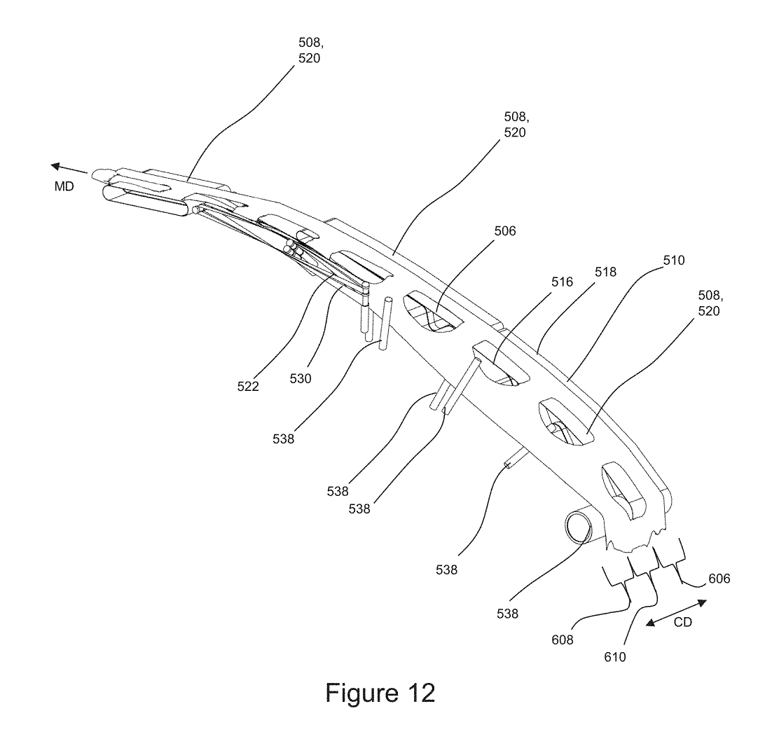

It is also to be appreciated that the folding apparatus 510 may be configured without a rail 540, thus, completely eliminating the frictional forces that would otherwise act on the central region 610 of the substrate 600 during the folding process. For example, as shown in FIG. 12, the folding apparatus 500 may be configured such that the folding axis 506 is completely defined by the first longitudinal edge 516 of the first conveyor 508.

In some configurations, the folding apparatus 500 is followed by a waist alignment unit that may include upper and/or lower vacuum conveyors. Such conveyors may pivot to provide a web steering function to align features of the folded and non-folded sides of the substrate, either relative to each other or relative to a reference value. Detection of the tracking position may be by dedicated sensor or a machine vision system. In some embodiments, edges of the substrate on the folded side and the non-folded side of an article are detected using an FR6001 sensor commercially available from Erhardt+Leimer. In some configurations, these sensors are mounted on a singled fixed bracket, and setpoint adjustments are made by changing a variable in the electronic controller. In some embodiments, the waist alignment unit may be replaced by a tracking device at one of the folding board rollers, which may include a camber roller replacing one or more of the folding rollers 538. Such a camber roller may also be feedback controlled via a downstream sensor or vision system.

To maintain machine direction alignment of the folding and non-folding portions of the substrate, one of more elements of the drive system may have a controllable velocity. In some configurations, the speed of a downstream drive point, preferably a vacuum conveyor may be varied, while maintaining the folding conveyor and a downstream drive point for the non-folded side at a constant surface velocity. Such velocity control may be open loop, but may also be accomplished by closed loop feedback control based upon signals from downstream sensors or machine vision systems. An example of such a feedback control system is a Proportional-Integral-Derivative (PID) controller, optionally with feed-forward and speed compensation, such as is implemented in common industrial controllers, such as the ControLogix platform from Rockwell Automation. The input signal may also include a position offset between folded and unfolded features on one or a series of articles. The input signal may also be a time difference measured between features on folded and unfolded portions of one or a series of articles. It is also to be appreciated that the machine direction alignment of features on folded and unfolded portions of one or a series of articles may also be accomplished by varying the path length of either the folded or unfolded portion of the substrate.

As mentioned above, the substrate 600 may be delivered flat or substantially flat to the folding apparatus 500. In some configurations, opposed pivoting camber rollers track and spread the substrate prior to folding. In some configurations, the tracking function and spreading functions are feed-back controlled, using commercially available web guides. In some configurations, the sensors of these web guides may be connected to a quality monitoring system, the edge positions of each side of the substrate may be stored, and/or the setpoints and control parameters may be remotely adjusted through an electronic controller. The folding centerline may be set by the cross-direction position of the substrate as the substrate enters the folder.

This application claims the benefit of U.S. Provisional Application No. 62/324,954 filed on Apr. 20, 2016, the entirety of which is incorporated by reference herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of folding absorbent articles, the method comprising the steps of:
advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction;
conveying the first outer region from an upstream end region of a first conveyor to a downstream end region of the first conveyor, the first conveyor comprising a first longitudinal edge separated from a second longitudinal edge in the cross direction;
partially folding the central region of the substrate along the first longitudinal edge of the first conveyor;
advancing the first surface of the partially folded central region to a rail adjacent the first longitudinal edge of the first conveyor, the rail extending for a length in the machine direction;
conveying the partially folded central region of the substrate with a second conveyor, wherein the second conveyor comprises a carrier surface comprising an upstream end region angularly offset from a downstream end region to define a generally helical-shaped conveyance path along the machine direction, the carrier surface contacting the second surface of the central region while advancing the first surface of the central region along the length of the rail; and
folding the central region around the rail to position the second outer region into a facing relationship with the first outer region.

2. The method of claim 1, further comprising the step of applying a vacuum force to the substrate with the second conveyor.

3. The method of claim 1, wherein the step of partially folding the central region of the substrate further comprises partially folding the substrate such that the second outer region is separated from first outer region by an angle of about 90 degrees.

4. The method of claim 1, further comprising the step of conveying the partially folded central region of the substrate with a third conveyor, wherein the third conveyor comprises a carrier surface comprising an upstream end region angularly offset from a downstream end region to define a generally helical-shaped conveyance path along the machine direction, the carrier surface contacting the second surface of the second outer region while advancing the first surface of the central region along the length of the rail.

5. The method of claim 4, wherein the carrier surface of the third conveyor also contacts the second surface of the central region.

6. The method of claim 1, wherein the first conveyor comprises a belt extending along a curved path from the upstream end region to the downstream end region.

7. The method of claim 1, further comprising the step of applying a vacuum force to the substrate with the first conveyor.

8. The method of claim 1, wherein the step of partially folding the central region along the first longitudinal edge of the first conveyor further comprises conveying the second outer region along a plurality of rollers, each roller having a different angular orientation relative to the first longitudinal edge of the first conveyor to define a substantially helical-shaped web path.

9. The method of claim 1, wherein the first outer region comprises a first continuous elastic belt, the second outer region comprises a second continuous elastic belt; and the central region comprises a plurality of absorbent cores spaced apart from each other along the machine direction.

10. The method of claim 9, further comprising the step of the cutting holes through the substrate, wherein the holes are spaced apart from each other along the machine direction in the central region.

11. A method of folding absorbent articles, the method comprising the steps of:
advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction;
conveying the first outer region from an upstream end region of a first conveyor to a downstream end region of the first conveyor, wherein the first conveyor comprises a first carrier surface, the first carrier surface contacting the first surface of the first outer region of the substrate;
wrapping a portion of the first surface of the central region of the substrate around a rail, the rail extending for a length in the machine direction;
conveying the central region of the substrate with a second conveyor, wherein the second conveyor comprises a second carrier surface comprising an upstream end region angularly offset from a downstream end region to define a generally helical-shaped conveyance path along the machine direction, the second carrier surface contacting the second surface of the central region while advancing the wrapped portion of the substrate along the length of the rail;
conveying the second outer region of the substrate with a third conveyor, wherein the third conveyor comprises a carrier surface comprising an upstream end region angularly offset from a downstream end region to define a generally helical-shaped conveyance path along the machine direction, the carrier surface contacting the second surface of the second outer region while advancing the conveying the central region of the substrate with the second conveyor; and
folding the substrate around the rail to position the second outer region into a facing relationship with the first outer region.

12. The method of claim 11, further comprising the step of applying a vacuum force to the substrate with the second conveyor.

13. The method of claim 11, wherein the step of wrapping the portion of the substrate further comprises partially folding the substrate such that the second outer region is separated from first outer region by an angle of about 90 degrees.

14. The method of claim 11, further comprising the step of applying a vacuum force to the substrate with the third conveyor.

15. The method of claim 14, wherein the carrier surface of the third conveyor also contacts the second surface of the central region.

16. The method of claim 11, wherein the first conveyor comprises a belt extending along a curved path from the upstream end region to the downstream end region.

17. The method of claim 11, further comprising the step of applying a vacuum force to the substrate with the first conveyor.

18. The method of claim 11, wherein the step of wrapping a portion of the first surface of the central region further comprises conveying the second outer region along a plurality of rollers, each roller having a different angular orientation relative to the first longitudinal edge of the first conveyor to define a substantially helical-shaped web path.

19. A method of folding absorbent articles, the method comprising the steps of:
advancing a substrate in a machine direction, the substrate comprising a first surface and an opposing second surface, the substrate further comprising a first outer region separated from a second outer region in a cross direction by a central region, wherein the first outer region and the second outer region are continuous in the machine direction and wherein the central region is discontinuous in the machine direction;
conveying the first outer region from an upstream end region of a first conveyor to a downstream end region of the first conveyor, wherein the first conveyor comprises a first carrier surface, the first carrier surface contacting the first surface of the first outer region of the substrate;
applying a vacuum force to the central region of the substrate with a second conveyor, wherein the second conveyor comprises a second carrier surface comprising an upstream end region angularly offset from a downstream end region to define a generally helical-shaped conveyance path along the machine direction;
applying a vacuum force to the second outer region of the substrate with a third conveyor, wherein the third conveyor comprises a third carrier surface comprising an upstream end region angularly offset from a downstream end region to define a generally helical-shaped conveyance path along the machine direction;
folding the substrate to position the second outer region into a facing relationship with the first outer region by advancing the central region and second outer region of the substrate with the second and third conveyors in the machine direction.

20. The method of claim 19, wherein the first outer region comprises a first continuous elastic belt, the second outer region comprises a second continuous elastic belt; and the central region comprises a plurality of chassis spaced apart from each other along the machine direction.
